(19) United States
(12) Patent Application Publication
(10) Pub. No.: US 2024/0399122 A1
(43) Pub. Date: Dec. 5, 2024

(54) SYSTEMS, DEVICES, AND METHODS FOR MAINTAINING FLOW IN ADJUSTABLE SHUNTING SYSTEMS

(71) Applicant: Shifamed Holdings, LLC, Campbell, CA (US)

(72) Inventors: Tessa Bronez, Campbell, CA (US); Shahla Nemati, Campbell, CA (US); Tom Saul, Portland, OR (US); Eric Schultz, Los Altos, CA (US); David Batten, San Francisco, CA (US)

(21) Appl. No.: 18/698,338

(22) PCT Filed: Oct. 13, 2022
(86) PCT No.: PCT/US2022/046604

§ 371 Date: Apr. 3, 2024

Related U.S. Application Data
(60) Provisional application No. 63/255,379, filed on Oct. 13, 2021.
(60) Provisional application No. 63/340,825, filed on May 11, 2022.

(51) Int. Cl. A61M 27/00

(52) U.S. Cl. CPC A61M 27/002; A61M 2205/0216; A61M 2205/0244; A61M 2205/0266; A61M 2205/04; A61M 2205/3331; A61M 2205/7545

(57) ABSTRACT

The present technology is generally directed to an adjustable shunting system for draining fluid from a first body region to a second body region. The adjustable shunting system can include a screen assembly configured to at least partially prevent debris from entering an internal portion of the adjustable shunting system. For example, the screen can be at least partially aligned with one or more fluid inlets of the adjustable shunting system. In some embodiments, the adjustable shunting system include one or more actuators that can be actuated via energy. In such embodiments, the screen assembly can be configured such that the actuators are accessible to energy through the screen. In these and other embodiments, the screen can be at least partially cleaned of debris by applying non-invasive ablative energy to the screen.

### CROSS-REFERENCE TO RELATED APPLICATION(S)

The present application claims priority to U.S. Provisional Patent Application No. 63/255,379, filed Oct. 13, 2021, and U.S. Provisional Patent Application No. 63/340,825, filed May 11, 2022, the disclosures of which are incorporated by reference herein in their entireties.

### TECHNICAL FIELD

The present technology generally relates to implantable medical devices and, in particular, to adjustable shunting systems and associated methods for selectively controlling fluid flow between a first body region and a second body region of a patient.

### BACKGROUND

Implantable shunting systems are widely used to treat a variety of patient conditions by shunting fluid from a first body region/cavity to a second body region/cavity. The flow of fluid through the shunting systems is primarily controlled by the pressure gradient across the shunt and the physical characteristics of the flow path defined through the shunt (e.g., the resistance of the shunt lumen(s)). However, most shunting systems have a single static flow path that is not adjustable. Accordingly, one challenge with conventional shunting systems is selecting the appropriate size shunt for a particular patient. A shunt that is too small may not provide enough therapy to the patient, while a shunt that is too large may create new issues in the patient. Despite this, most conventional shunts cannot be adjusted after implantation and, therefore, cannot be adjusted or titrated to meet the patient's individual and variable needs and/or to account for changes in flow-related characteristics, such as flow volume, inflow pressure, and/or outflow resistance.

### BRIEF DESCRIPTION OF THE DRAWINGS

Many aspects of the present technology can be better understood with reference to the following drawings. The components in the drawings are not necessarily drawn to scale. Instead, emphasis is placed on illustrating clearly the principles of the present technology. Furthermore, components can be shown as transparent in certain views for clarity of illustration only and not to indicate that the component is necessarily transparent. Components may also be shown schematically.

FIG. 1A is a perspective view of an adjustable shunting system including a screen element configured in accordance with various embodiments of the present technology.

FIG. 1B is a top view of the adjustable shunting system of FIG. 1A.

FIG. 1C is an enlarged top view of select aspects of the adjustable shunting system of FIG. 1B with other aspects omitted for the purpose of clarity.

FIG. 2 is a top view of the screen assembly of FIG. 1A and select aspects of the system of FIG. 1A, with other aspects of the system omitted for the purpose of clarity.

FIG. 3 is a block diagram illustrating a method of operating an adjustable shunting system having a screen assembly and configured in accordance with various embodiments of the present technology.

FIGS. 4A and 4B are top and cross-sectional views, respectively, of an adjustable shunting system including a screen assembly and configured in accordance with additional embodiments of the present technology.

FIG. 4C is an enlarged cross-sectional view of a portion of the adjustable shunting system of FIG. 4B.

FIGS. 5A and 5B are top and cross-sectional views, respectively, of another adjustable shunting system including a screen assembly and configured in accordance with various embodiments of the present technology.

FIG. 6A is a top view, and FIGS. 6B and 6C are partially-exploded isometric views of an adjustable shunting system including screen assemblies and configured in accordance with further embodiments of the present technology.

FIGS. 7A and 7B are a top view and an end view, respectively, of select aspects of an adjustable shunting system configured in accordance with embodiments of the present technology, with other aspects of the system omitted for illustrative clarity.

FIGS. 8A and 8B are a top view and a bottom view, respectively, of an adjustable shunting system configured in accordance with embodiments of the present technology.

### DETAILED DESCRIPTION

The present technology is generally directed to systems, devices, and methods for maintaining flow in adjustable shunting systems. At least some of the adjustable shunting systems described herein can include screen assemblies configured to filter aqueous passing through the shunting system and at least partially or fully prevent debris from entering an internal portion (e.g., the fluid inlets, a plate assembly, one or more channels, etc.) of the shunting system. The disclosed screen assemblies can include a plurality of pores or openings formed therein, with the individual pores being sized to at least partially prevent debris or other contaminants from entering the internal portion of the shunting system while also at least partially allowing fluid (e.g., aqueous) to flow through the internal portion of the shunting system. Additionally, the screen assemblies can be configured such that non-invasive energy (e.g., ablative laser energy) can be applied to the screen to at least partially dissolve, burn-off, or otherwise remove the captured debris.

As described in greater detail below, it is expected that the present technology may exhibit one or more advantageous characteristics that improve operation of adjustable shunting systems. Many adjustable shunting systems include channels, lumens, or other paths through which fluid can flow from a first body region to a second body region. The fluid that flows through these systems can include cellular matter, particulate matter, and/or other debris that can partially or fully obstruct (e.g., block, clog, etc.) the fluid flow paths through these systems. Thus, the operation of many adjustable shunting systems can be adversely affected (e.g., rendered partially or fully inoperable) due to any such debris that enters these systems. Additionally, it can be difficult to remove or clean such debris from many adjustable shunting systems without first removing these systems from a patient. Furthermore, some adjustable shunting systems include actuators configured to provide an adjustable therapy to a patient; previous attempts to reduce or prevent debris from entering such adjustable shunting systems can interfere with the operation of the actuators of these systems. Accordingly, compared with conventional systems, adjustable shunting systems including screen assemblies configured in accordance with embodiments of the present technology are expected to provide an adjustable therapy to patients while inhibiting and/or at least partially preventing debris/contaminants from adversely affecting operation of such systems.

In some embodiments, the screen assemblies can include one or more sealing elements configured to sealingly engage at least a portion of the adjustable shunting system. The sealing elements are expected to further reduce the likelihood that debris will block or clog the adjustable shunting systems. Additionally, or alternatively, the adjustable shunting systems can include a plurality of fluid inlets, and the screen assemblies (e.g., one or more pores of the screen assemblies) can be positioned above and/or cover the plurality of fluid inlets to define a fluid space between the screen assemblies and individual ones of the plurality of fluid inlets. During operation, the fluid space is expected to allow fluid to reach individual ones of the plurality of fluid inlets if the screen assembly becomes partially blocked or clogged.

At least some of the adjustable shunting systems described herein include a plurality of fluid inlets configured to reduce or prevent tissue ingrowth, which, in turn, is expected to further reduce the likelihood that the adjustable shunting systems become blocked or clogged. Additionally, or alternatively, at least some of the adjustable shunting systems include one or more channels having varying dimensions, such as a channel having a first width at a first end of the channel and a second width at a second end of the channel. The second width can be greater than the first width: fluid (e.g., aqueous) can flow through the channel from the first end to the second end, such that the width of the channel can increase in the direction of the fluid flow through the channel. In some aspects, channels with varying dimensions are expected to be less likely to become blocked or clogged. In these and other embodiments, individual ones of the channels can be fluidly coupled to a plurality of inlets, such as two or more inlets arranged in series along the length of a channel. If a portion of the channel becomes blocked or clogged, an inlet downstream from the blockage/clog can be opened to bypass the blockage/clog and/or otherwise allow flow through the channel to resume.

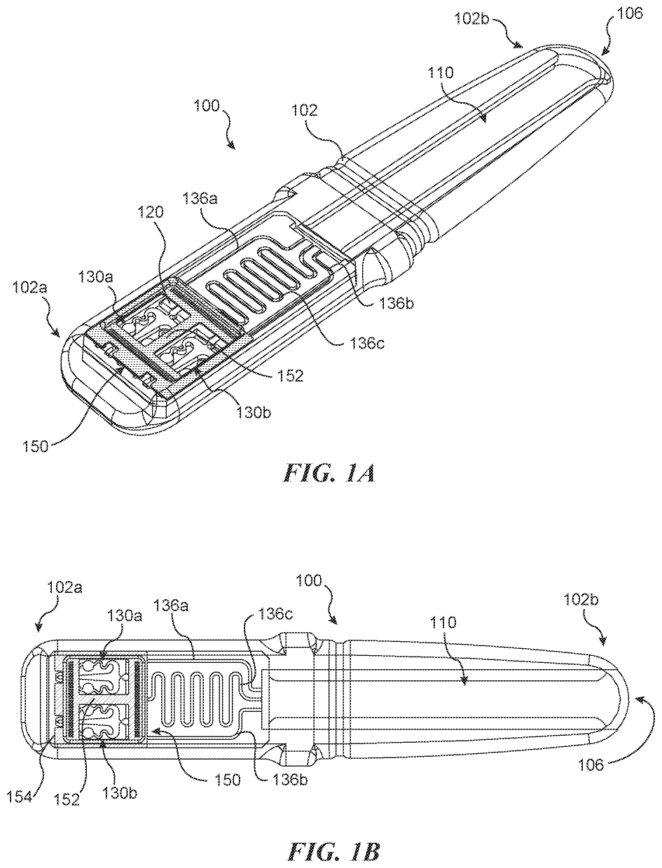

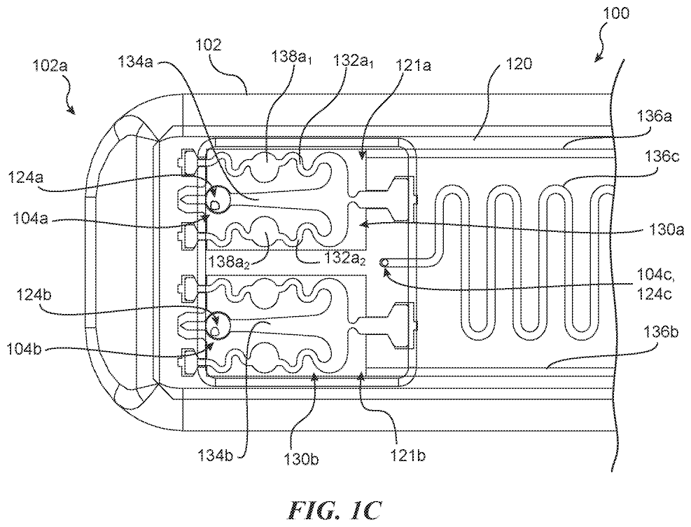

The terminology used in the description presented herein is intended to be interpreted in its broadest reasonable manner, even though it is being used in conjunction with a detailed description of certain specific embodiments of the present technology. Certain terms may even be emphasized below: however, any terminology intended to be interpreted in any restricted manner will be overtly and specifically defined as such in this Detailed Description section. Additionally, the present technology can include other embodiments that are within the scope of the examples but are not described in detail with respect to FIGS. 1A-8B.

Reference throughout this specification to “one embodiment” or “an embodiment” means that a particular feature, structure, or characteristic described in connection with the embodiment is included in at least one embodiment of the present technology. Thus, the appearances of the phrases “in one embodiment” or “in an embodiment” in various places throughout this specification are not necessarily all referring to the same embodiment. Furthermore, the particular features or characteristics described herein may be combined in any suitable manner in one or more embodiments.

Reference throughout this specification to relative terms such as, for example, “generally,” “approximately,” and “about” are used herein to mean the stated value plus or minus 10%. Reference throughout this specification to the term “resistance” refers to fluid resistance unless the context clearly dictates otherwise. The terms “drainage rate” and “flow rate” are used interchangeably to describe the movement of fluid through a structure at a particular volumetric rate. The term “flow” is used herein to refer to the motion of fluid, in general.

Although certain embodiments of adjustable shunting system are described in terms of shunting fluid from an anterior chamber of an eye, one of skill in the art will appreciate adjustable shunting systems having screen elements configured in accordance with embodiments of the present technology can be readily adapted to shunt fluid from and/or between other portions of the eye, or, more generally, from and/or between a first body region and a second body region. Moreover, while the certain embodiments herein are described in the context of glaucoma treatment, any of the embodiments herein, including those referred to as “glaucoma shunts” or “glaucoma devices” may nevertheless be used and/or modified to treat other diseases or conditions, including other diseases or conditions of the eye or other body regions. For example, the systems described herein can be used to treat diseases characterized by increased pressure and/or fluid build-up, including but not limited to heart failure (e.g., heart failure with preserved ejection fraction, heart failure with reduced ejection fraction, etc.), pulmonary failure, renal failure, hydrocephalus, and the like. Moreover, while generally described in terms of shunting aqueous, the systems described herein may be applied equally to shunting other fluid, such as blood or cerebrospinal fluid, between the first body region and the second body region.

FIGS. 1A and 1B illustrate an adjustable shunting system 100 (“the system 100”) including a screen assembly 150 configured in accordance with various embodiments of the present technology. Specifically, FIG. 1A illustrates a perspective view of the system 100 and FIG. 1B illustrates a top view of the system 100. As described in greater detail below, the screen assembly 150 is configured to filter aqueous passing through the system 100 and at least partially or fully prevent debris from entering an internal portion (e.g., the fluid inlets, a plate assembly, one or more channels, etc.) of the system 100.

Referring to FIGS. 1A and 1B together, the system 100 includes a generally elongated housing 102 (“the housing 102”) and a plate assembly or cartridge 120. The housing 102 (which can also be referred to as a casing, membrane, shunting element, or the like) extends between a first end portion 102a and a second end portion 102b. The housing 102 can include one or more openings (e.g., the opening 104 of FIG. 1C) configured to receive fluid (e.g., aqueous) from an environment (e.g., the first body region) external to the system 100. In the illustrated embodiment, the system 100 includes one or more fluid outlets 106 positioned proximate the second end portion 102b of the housing 102. In other embodiments, the one or more fluid outlets 106 can have any other suitable position relative to the housing 102. The housing 102 further includes a main fluid conduit 110 fluidly coupling the plate assembly 120 to the one or more fluid outlets 106. The housing 102 can also optionally have one or more wings or appendages (not shown) having holes (e.g., suture holes) or other attachment features for securing the elongated housing 102 in a desired position. The housing 102 can be composed of a slightly elastic or flexible biocompatible material (e.g., silicone, etc.).

The plate assembly 120 (which can also be referred to as a flow control plate, a flow control cartridge, a plate structure, or the like) is positioned at least partially or fully within the housing 102. In the illustrated embodiment, for example, the housing 102 substantially and/or fully encases the plate assembly 120 at or proximate the first end portion 102a of the housing 102. In other embodiments, the plate assembly 120 can have any other suitable position at least partially or fully within the housing 102. The plate assembly 120 can be fluidly coupled to the one or more fluid outlets 106 via the main fluid conduit 110, and can be configured to control the flow of fluid through the system 100. In some embodiments, an upper surface of the plate assembly 120 forms a substantial fluid seal with an interior surface of the elongated housing 102 at the first end portion 102a such that the only way for fluid to enter the system 100 is through the fluid inlets 124. Accordingly, for fluid to flow through the system 100, it generally must flow through the plate assembly 120. As described in greater detail below and with reference to FIG. 1C, the plate assembly 120 can include: (i) one or more fluid apertures or inlets (e.g., the fluid inlets 124a-c of FIG. 1C) that align with the opening 104 (FIG. 1C) in the elongated housing 102 and allow fluid to enter the plate assembly 120; (ii) one or more actuators 130a-b (“the actuators 130”), each of the actuators 130 positioned to control the flow of fluid through a corresponding one of the fluid inlets 124a-c; and/or (iii) one or more channels 136a-c through which fluid (e.g., aqueous) can flow through the system 100 (e.g., from the one or more fluid inlets 124a-c to and/or toward the one or more fluid outlets 106).

In the illustrated embodiment, the screen assembly 150 is positioned proximate the first end portion 102a of the housing 102. In other embodiments, however, the screen assembly 150 can be positioned proximate the second end portion 102b of the housing 102, or can have any other suitable position relative to the housing 102 of the system 100. The screen assembly 150 can include a screen or filter 152 (“the screen 152”). At least part of the screen 152 can be aligned with at least a portion (e.g., the opening 104, the plate assembly 120, the actuators 130, etc.) of the system 100. In the illustrated embodiment, for example, the screen 152 is at least partially aligned with (e.g., positioned above) the plate assembly 120 and the actuators 130. Continuing with this example, the screen 152 is positioned within the housing 102, at least partially between the housing 102 and the plate assembly 120 with at least part of the screen 152 aligned with (e.g., covering, positioned above, etc.) the one or more fluid inlets 124a-c (FIG. 1C), such that fluid from the first body region can pass through the screen 152 before entering the plate assembly 120 via the fluid inlets 124. Accordingly, the screen 152 can protect at least one, a plurality, or all of the fluid inlets 124 of the system 100 from debris and/or other matter. In other embodiments, the screen 152 can be positioned at least partially or fully external to the housing 102, such as above or at least partially within the opening 104 of the housing 102. In further embodiments, at least a portion of the screen 152 can be positioned within the plate assembly 120, such as between one or more of the fluid inlets 124 and the actuators 130 and/or between the actuators 130 and one or more of the channels 136, such that fluid that enters the plate assembly 120 can pass through the screen 152 before entering one or more of the channels 136. In still further embodiments, the screen 152 can have any other suitable position relative to the system 100. Although described as having a single screen 152, in other embodiments the screen assembly 150 can include more screens, such as at least two, three, four, or any other suitable number of screens, each of which can have any suitable position relative to the system 100.

The screen 152 can be formed from silicon, acrylic, a shape-memory material, and/or any other suitable material. In at least some embodiments, for example, the screen 152 is formed from polydimethylsiloxane (PDMS), polydimethylacrylamide (PDMA), and/or super-elastic nitinol. In FIGS. 1A and 1B, select portions of the screen 152 are illustrated as being opaque to better illustrate aspects of the present technology. It will be appreciated that, in other embodiments, all or part of the screen 152 can be transparent, translucent (e.g., partially transparent), opaque, or have any other suitable transmissivity.

In operation, the system 100 is configured to provide an adjustable therapy for draining fluid from a first body region to a second, different body region, such as to drain aqueous from an anterior chamber of a patient's eye. The plate assembly 120 is configured to selectively control the flow of fluid entering the system 100. As described in greater detail below and with reference to FIG. 1C, each of the actuators 130 can be actuated (via, e.g., energy) to control the flow of fluid through a corresponding fluid inlet. In some embodiments, the fluid (e.g., aqueous) can include one or more cellular masses, particulate matter, and/or any other debris in the first body region. Accordingly, screens in accordance with embodiments of the present technology, such as the screen 152, can be configured: (i) to allow a first portion of the fluid to flow through at least an internal portion the system 100 (e.g., the fluid inlets 124, the plate assembly 120, the chambers 121, the channels 136, etc.); (ii) to at least partially prevent a second portion of the fluid (e.g., debris) from partially or fully clogging, obstructing, blocking, or otherwise preventing flow of the first portion of the fluid through the internal portion of the system 100; (iii) to be least partially cleaned or unclogged by application of a first non-invasive energy (e.g., laser energy); and/or (iv) to allow the actuators 130 to be accessible (e.g., through at least part of the screen 152) to second non-invasive energy (e.g., laser energy) to actuate one or more of the actuators 130.

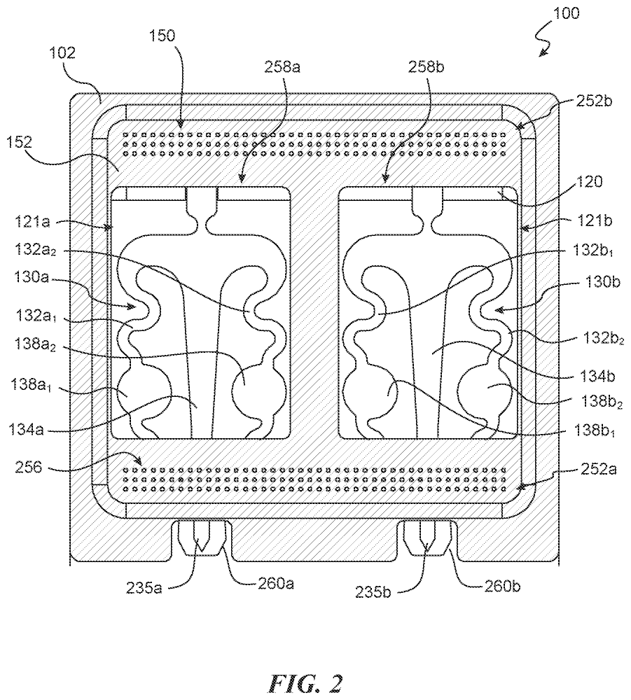

As a specific example, the screen 152 can allow aqueous from the first body region to enter the system 100 while at least partially filtering or screening one or more cellular masses and/or other debris in the first body region (e.g., suspended in the aqueous) from entering the internal portion of the system 100. Continuing with the example, during operation, the cellular masses and/or other debris can accumulate on and/or within the screen 152 such that the screen 152 can become at least partially blocked or clogged by the cellular masses and/or other debris. Unlikely many conventional adjustable shunting systems, adjustable shunting systems including screen assemblies configured in accordance with embodiments of the present technology can be at least partially cleaned or unclogged via delivery of non-invasive energy and without being removed from a patient. For example, as described in greater detail below referring to FIG. 2, energy (e.g., laser energy) can be applied to the screen 152 to partially or fully unblock, unclog, or otherwise remove (e.g., dissolve, burn-off, etc.) part or all of the cellular masses and/or the debris captured, filtered, or otherwise screened by the screen 152.

FIG. 1C illustrates an enlarged top view of a portion the system 100 with the screen assembly 150 omitted to better illustrate other aspects of the system 100. Referring FIG. 1C and as described previously, the housing 102 can include an opening 104 configured to receive fluid (e.g., aqueous) from an environment external to the system 100 (e.g., the first body region). The opening 104 can be aligned with one or more fluid inlets 124 in the plate assembly 120. In the illustrated embodiment, for example, the opening 104 is aligned with a first fluid inlet 124a, a second fluid inlet 124b, and a third fluid inlet 124c. The fluid inlets 124 permit fluid to enter an interior of the plate assembly 120 (and thus an interior of the elongated housing 102) from an environment external to the system 100. Although not shown in FIG. 1C, it can be appreciated that the screen 152 can be aligned with one or more of the fluid inlets 124 such that, before flowing through the plate assembly 120, fluid can flow through the screen 152 (e.g., as described previously and with reference to FIGS. 1A and 1B).

The fluid path through the plate assembly 120 depends on which fluid inlet 124 the fluid enters through. For example, fluid that enters the system 100 via the first fluid inlet 124a flows into a first chamber 121a of the plate assembly 120 and drains to the main fluid conduit 110 via the first channel 136a. Fluid that enters the system 100 via the second fluid inlet 124b flows into a second chamber 121b of the plate assembly 120 and drains to the main fluid conduit 110 via a second channel 136b. Fluid that enters the system 100 via the third fluid inlet 124c drains to the main fluid conduit 110 via the third channel 136c. The chambers 121a-b and the channels 136a-c can be fluidly isolated such that there are three discrete flow paths through the plate assembly 120. The channels 136a-c can also have different geometric configurations (e.g., lengths) relative to one another such that they have different fluid resistances and thus, can provide different flow rates.

The relative level of therapy provided by each fluid path can be different so that a user may adjust/modulate the level of therapy provided by the system 100 by selectively opening and/or closing various fluid paths (e.g., by selectively interfering with or permitting flow through individual fluid inlets 124). For example, (i) the first fluid inlet 124a and/or the first channel 136a can provide a first fluid resistance when fluid travels primarily therethrough: (ii) the second fluid inlet 124b and/or the second channel 136b can provide a second fluid resistance less than the first fluid resistance when fluid travels primarily through the second fluid inlet 124b; and (iii) the third fluid inlet 124c and/or the third channel 136c can provide a third fluid resistance less than the first fluid resistance when fluid travels primarily through the third fluid inlet 124c. Continuing with this example, under a given pressure, each of the fluid inlets 124a-c can provide a different fluid flow rate through the system 100. In other embodiments, the channels 136a-c can have the same or generally the same geometric configurations such that they have the same or generally the same fluid resistances, and thus provide similar flow rates for a given pressure.

In the illustrated embodiment, the first actuator 130a is positioned in the first chamber 121a and configured to control the flow of fluid through the first fluid inlet 124a and the second actuator 130b is positioned in the second chamber 121b and configured to control the flow of fluid through the second fluid inlet 124b. The first actuator 130a can include a first projection or gating element 134a configured to moveably interface with the first fluid inlet 124a, e.g., to move between a first (e.g., “open”) position in which the gating element 134a does not substantially prevent fluid from flowing through the first fluid inlet 124a (e.g., by being offset from and/or otherwise not interfering with the first fluid inlet 124a) and a second (e.g., “closed”) position in which the gating element 134a substantially prevent fluid from flowing through the first fluid inlet 124a (e.g., by blocking, being positioned within, and/or otherwise aligned with the first fluid inlet 124a). In some embodiments, the gating element 134a can be configured to move to one or more intermediate positions between the first (e.g., open) and the second (e.g., closed) position. The second actuator 130b can include a second gating element 134b that operates in a similar manner as the gating element 134a (e.g., moveable between open and closed positions relative to the second fluid inlet 124b). In the illustrated embodiment, the flow of fluid through the third inlet 124c is not controlled by an actuator, and the third inlet 124c and/or the third channel 136 can be configured to provide a constant or minimum flow rate of fluid through the system 100. In other embodiments, however, the plate assembly 120 can include a third actuator generally similar to or the same as the first actuator 130a and/or the second actuator 130b, and the third actuator can have a third gating element that operates in a similar manner as the first gating element 134a and/or the second gating element 134b (e.g., moveable between open and closed positions relative to the third fluid inlet 124c). In such embodiments, the third actuator can be positioned in a third chamber generally similar to or the same as the first chamber 121a and/or the second chamber 121b.

The first actuator 130a can further include a first actuation element 132a₁ and a second actuation element 132a₂ that drive movement of the gating element 134a between the first (e.g., open) position and the second (e.g., closed) position. The first actuation element 132a₁ and the second actuation element 132a₂ can be composed at least partially of a shape memory material or alloy (e.g., nitinol). Accordingly, the first actuation element 132a₁ and the second actuation element 132a₂ can be transitionable at least between a first material phase or state (e.g., a martensitic state, a R-phase, a composite state between martensitic and R-phase, etc.) and a second material phase or state (e.g., an austenitic state, an R-phase state, a composite state between austenitic and R-phase, etc.). In the first material state, the first actuation element 132a₁ and the second actuation element 132a₂ may have reduced (e.g., relatively less stiff) mechanical properties that cause the actuation elements to be more easily deformable (e.g., compressible, expandable, etc.) relative to when the actuation elements are in the first material state. In the second material state, the first actuation element 132a₁ and the second actuation element 132a₂ may have increased (e.g., relatively stiffer) mechanical properties relative to the first material state, causing an increased preference toward a specific preferred geometry (e.g., original geometry, manufactured or fabricated geometry, heat set geometry, etc.). The first actuation element 132a₁ and the second actuation element 132a₂ can be selectively and independently transitioned between the first material state and the second material state by applying energy (e.g., laser energy, electrical energy, etc.) to the first actuation element 132a₁ or the second actuation element 132a₂ to heat it above a transition temperature (e.g., above an austenite finish (A_{f}) temperature, which is generally greater than body temperature). If the first actuation element 132a₁ (or the second actuation element 132a₂) is deformed relative to its preferred geometry when heated above the transition temperature, the first actuation element 132a₁ (or the second actuation element 132a₂) will move to and/or toward its preferred geometry. In some embodiments, the first actuation element 132a₁ and the second actuation element 132a₂ are operably coupled such that, when the actuated actuation element (e.g., the first actuation element 132a₁) transitions toward its preferred geometry, the non-actuated actuation element (e.g., the second actuation element 132a₂) is further deformed relative to its preferred geometry.

The first actuation element 132a₁ and the second actuation element 132a₂ generally act in opposition. For example, the first actuation element 132a₁ can be actuated to move the gating element 134a to and/or toward the first (e.g., open) position, and the second actuation element 132a₂ can be actuated to move the gating element 134a to and/or toward the second (e.g., closed) position. Additionally, as described above, the first actuation element 132a₁ and the second actuation element 132a₂ can be coupled such that as one moves toward its preferred geometry upon material phase transition, the other is deformed relative to its preferred geometry. This enables the actuation elements 132a to be repeatedly actuated and the gating element 134a to be repeatedly cycled between the first (e.g., open) position and the second (e.g., closed) position.

In some embodiments, each actuation element 132 can include one or more targets 138. The target(s) 138 can be thermally coupled to the corresponding actuation element(s) 132 such that that energy (e.g., laser energy) received at the target(s) 138 can dissipate through the corresponding actuation element(s) 132 as heat. The target(s) 138 can therefore be selectively targeted with energy to actuate the actuation elements 132. For example, to actuate the first actuation element 132a₁, heat/energy can be applied to the first target 138a₁, such as from an energy source positioned external to the patient's eye (e.g., a laser). The heat applied to the first target 138a₁ spreads through at least a portion of the first actuation element 132a₁, which can heat the first actuation element 132a₁ above its transition temperature. To actuate the second actuation element 132a₂ heat/energy can be applied to the second target 138a₂. The heat applied to the second target 138a₂ spreads through the second actuation element 132a₂, which can heat at least the portion of the second actuation element 132a₂ above its transition temperature. In the illustrated embodiment, the targets 138 are positioned generally centrally along a length of each individual actuation element 132. In other embodiments, the targets 138 can be positioned at an end region of each individual actuation element 132. In some embodiments, the targets 138 are composed of a same material (e.g., nitinol) as the actuation elements 132. Without being bound by theory, the increased surface area of the targets 138 relative to the actuation elements 132 is expected in increase the ease and consistency by which the actuators 130 can be actuated using an energy source (e.g., a laser) positioned external to the body.

The second actuator 130b can also include a pair of opposing shape-memory actuators and operate in the same or similar fashion as the first actuator 130a. In embodiments where the flow of fluid through the third inlet 124c is controlled by a third actuator, the third actuator can also include a pair of opposing shape-memory actuators and operate in the same or similar fashion as the first actuator 130a and/or the second actuator 130b. Although the system 100 is depicted as having two actuators 130a-b in FIGS. 1A-1C, in other embodiments the system 100 can include more or fewer actuators 130. In at least some embodiments, for example, the system 100 can include one, three, four, five, six, seven, eight, nine, ten, or any other suitable number of actuators 130. Although the system 100 is depicted as having three channels 136a-c in FIGS. 1A-IC, in other embodiments the system 100 can include more or fewer channels 136. In at least some embodiments, for example, the system 100 can include one, two, four, five, six, seven, eight, nine, ten, or any other suitable number of channels 136. In some embodiments, the number of channels 136 corresponds to the number of fluid inlets 124 and/or the number of actuators 130 in the system. Additional details regarding the operation of shape memory actuators, as well as adjustable glaucoma shunts, are described in U.S. Pat. Nos. 11,291,585 and 11,166,849, and International Patent Application Nos. PCT/US20/55144, PCT/US20/55141, PCT/US21/14774, PCT/US21/18601, PCT/US21/23238, and PCT/US21/27742, the disclosures of which are incorporated by reference herein in their entireties and for all purposes.

The energy (e.g., “actuating energy”) used to actuate the actuation elements can have generally similar or the same properties (e.g., optical properties, such as a generally similar or the wavelength and/or amplitude) as the energy (e.g., “cleaning energy”) used to at least partially clean or unclog the screen 152. It at least some embodiments, for example, the actuating energy and the cleaning energy can be the same, such that the energy used to clean the screen 152 can also be used to actuate the actuators 130. In other embodiments, however, the actuating energy and the cleaning energy can have different properties (e.g., optical properties, such as different wavelengths and/or amplitudes).

FIG. 2 is a top view of the screen assembly 150 and select aspects of the system 100 of FIGS. 1A-1C, with other aspects of the system 100 omitted for the purpose of clarity. The screen 152 of the screen assembly 150 can include a first end portion 252a and a second end portion 252b opposite the first end portion 252a. The first end portion 252a and/or the second end portion 252b of the screen 152 can include one or more screening or filtering elements 256 (“the screen elements 256”). One or more of the screening elements 256 can be at least partially aligned with (e.g., positioned above or below, positioned at least partially within, etc.) the opening 104 and/or at least one of the fluid inlets 124 described previously and with reference to FIGS. 1A-1C.

In the illustrated embodiment, each of the screening elements 256 is a circular hole or pore formed in the screen 152 and having a dimension (e.g., width, diameter, etc.) of about 10 μm. In other embodiments, each of the screening elements 256 can have an oval, square, pentagonal, hexagonal, curvilinear, rectilinear, and/or any other suitable shape. In these and other embodiments, each of the screening elements 256 can have a width of between about 0.1 μm to about 100 μm, such as at least 1 μm, 5 μm, 10 μm, 20 μm, 50 μm, and width therebetween, or another suitable width. Additionally, or alternatively, the configuration of individual ones of the screening elements 256 can be configured based, at least partially, on a flow resistance associated with the configuration. In at least some embodiments, for example, individual ones of the screening elements 256 can be configured to provide a flow resistance less than individual ones of the channels 136a-c (FIGS. 1A-1C). In further embodiments, the screening elements 256 can have any other suitable configuration. Although in the illustrated embodiment the first end portion 252a and the second end portion 252b of the screen 152 both include 126 screening elements, in other embodiments the first end portion 252a and/or the second end portion 252b can have more or fewer screening elements.

The screen 152 can further include one or more actuator access or viewing regions 258 (“the regions 258”). Each of the region 258 can extend partially or fully between the first end portion 252a and the second end portion 252b of the screen 152, and/or can be at least partially aligned with (e.g., positioned above) a corresponding one of the actuators 130 and/or the chambers 121. In the illustrated embodiment, for example, the screen 152 includes (i) a first region 258a aligned with at least part of the first actuator 130a and the first chamber 121a and (ii) a second region 258b aligned with at least part of the second actuator 130b and the second chamber 121b. Each of the regions 258 can be configured such that at least a portion of the corresponding actuators 130 (e.g., the actuation elements 132, the target 138, etc.) can be accessible to energy (e.g., laser energy) applied from outside the adjustable shunting system. In some embodiments, one or more of the regions 258 can be gaps or apertures in (e.g., formed in) the screen 152 and at least part (e.g., the actuator targets 138) of one or more of the actuators 130a-b can be accessible to energy through the corresponding regions 258 of the screen 152, such that energy directed toward one or more of the regions 258 can pass through the screen 152 and be incident on the correspond actuator(s) 130. In other embodiments, one or more of the regions 258 can be a portion of the screen 152 formed from a material (e.g., PDMS, PDMA, etc.) that is at least partially or fully transparent. In further embodiments, the entire screen 152 and/or screen assembly 150 can be formed from a material (e.g., PDMS, PDMA, etc.) that is at least partially or fully transparent. In these and other embodiments, the screen 152 can be formed a single sheet of material.

The screen assembly 150 can further include one or more indicator regions 260. Each of the indicator regions 260 can correspond to one of the actuators 130, such that each of the indicator regions 260 can be aligned with at least a portion of the corresponding actuator 130. In the illustrated embodiment, for example, the screen assembly 150 includes (i) a first indicator region 260a aligned with an end portion 235a of the first gating element 134a of the first actuator 130a and (ii) a second indicator region 260b aligned with an end portion 235b of the second gating element 134b of the second actuator 130b. Each of the indicator regions 260 can be at least partially or fully transparent, such that the corresponding end portions 235 can be visualized and/or observed (e.g., by a user and/or a practitioner of the system 100) through the corresponding indicator regions 260.

In operation, and as described previously, each of the screening elements 256 can be configured (i) to allow at least a first portion of the fluid to enter an internal portion (e.g., the fluid inlets 124, the plate assembly 120, the chambers 121, etc.) of the system 100 and (ii) to at least partially or fully prevent at least a second portion of the fluid (e.g., debris) from entering the internal portion of the system 100. In at least some embodiments, for example, each of the screening elements 256 can be sized such that the second portion of the fluid can be captured on and/or within one or more of the screening elements 256. This can at least partially prevent the second portion of the fluid from entering the internal portion of the system 100 Accordingly, during operation, one or more of the screening elements 256 may become at least partially or fully blocked or clogged by the debris filtered from the fluid. Energy (e.g., non-invasive energy, ablative laser energy, etc.) can be applied to the screening element(s) 256 to at least partially or fully unblock or unclog the screening element(s) 256. For example, the first end portion 252a and/or the second end portion 252b of the screen 152 can be configured to withstand exposure to energy such that the energy can be applied to the first end portion 252a and/or the second end portion 252b to partially or fully dissolve, burn-off, or otherwise remove debris from one or more of the screening elements 256. Additionally, as described previously, one or more of the regions 258 can be configured such that the corresponding actuator(s) 130 can be at least partially or fully accessible to energy (e.g., laser energy). For example, the first actuator 130a and/or second actuator 130b can be accessible to energy from outside the system 100 via the corresponding regions 258, such that the energy can be applied to selectively and/or individually actuate the actuators 130a-b. In some embodiments, before, during, and/or after one or more of the actuators 130 are actuated, one or more of the end portion 235 can be visualized via the corresponding indicator region 260 to determine whether the corresponding actuator 130 and/or gating element 134 is in the first (e.g., open) position or the second (e.g., closed configuration) position.

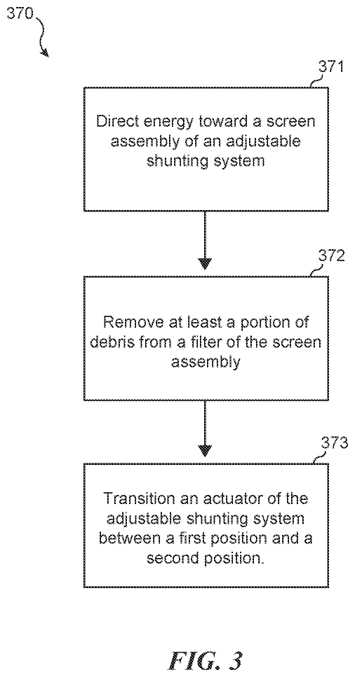

FIG. 3 is a flow diagram illustrating a method 370 of operating an adjustable shunting system configured in accordance with various embodiments of the present technology. The method 370 is illustrated as a set of blocks, steps, operations, or processes 371-373. All or a subset of the blocks 371-373 can be executed at least in part by various components of a system, such as the system 100 of FIGS. 1A-IC, filter assembly 150 of FIGS. 1A, 1B, and FIG. 2. For example, all or a subset of the blocks 371-373 can be executed at least in part by a screen assembly, a screen, a screening element, an actuator, an actuation assembly, and/or other portions of an adjustable shunting system. Additionally, or alternatively, all or a subset of the blocks 371-373 can be executed at least in part by an operator (e.g., a user, a patient, a caregiver, a family member, a physician, etc.) of the system. Furthermore, any one or more of the blocks 371-373 can be executed in accordance with the discussion above. Many of the blocks 371-373 of the method 370 are discussed in detail below with reference to FIGS. 1A-2 for the sake of clarity and understanding. It will be appreciated, however, that the method 370 may be used with other suitable adjustable shunting systems in addition to those described herein.

The method 370 begins at block 371 by directing energy toward a screen assembly of an adjustable shunting system. In some embodiments, for example, directing energy toward the screen assembly can include applying energy to a screen and/or one or more screening elements of the screen assembly. The screen and/or screening elements can be similar to the screens and/or screening elements discussed above with reference to FIGS. 1A-2. For example, applying the energy to a screen assembly can include applying energy to a first end portion 252a and/or a second end portion 252b of the screen 152, the first end portion 252a and/or the second end portion 252b including one or more screening elements 256, as described previously with reference to FIG. 2.

In these and other embodiments, directing energy toward the screen assembly can include directing energy toward one or more actuator access regions of the screen assembly. The one or more actuator access regions can be similar to the actuator access regions 258 discussed above with reference to FIG. 2. For example, directing the energy to the screen assembly can include applying the energy to one or more actuators 130 of the adjustable shunting system 100 via a corresponding one of the one or more actuator access regions 258, such that the energy passes through at least a portion of the screen assembly 150 (via, e.g., the access regions 258). In such embodiments, applying energy to the one or more actuators can include applying the energy to one or more targets 138 of the actuators 130 via the corresponding actuator access regions 258.

At block 372, the method 370 continues by removing at least a portion of any cellular masses, particulate matter, and/or any other debris from the screen of the screen assembly. In some embodiments, for example, removing at least the portion of the debris from the screen can include removing at least the portion of the debris from one or more screening elements of the screen. The screen and/or the screening elements can be similar to the screens 152 and/or screening elements 256 discussed above with reference to FIGS. 1A-2. For example, removing at least the portion of the debris from the screen 152 can include dissolving and/or burning off one or more cellular masses and/or any other debris captured on and/or at least partially within one or more of the screening elements 256 of the screen 152.

At block 373, the method 370 continues by transitioning an actuator of the adjustable shunting system between a first position and a second position. In some embodiments, for example, transitioning the actuator between the first position and the second position can include (i) moving an actuation element of the actuator to and/or toward a preferred geometry of the actuation element or (ii) deforming the actuation element relative to the preferred geometry. The actuator and the actuation element can be generally similar to the actuators 130 and actuation elements 132 described previously with reference to FIGS. 1A-2. For example, transitioning the actuator 130 between the first position and the second position can include (i) moving a first actuation element 132a₁ of a first actuator 130a to and/or toward a preferred geometry or (ii) deforming the first actuation element 132a₁ relative to the preferred geometry.

Although the steps of the method 370) are discussed and illustrated in a particular order, the method 370 illustrated in FIG. 3 is not so limited. In other embodiments, the method 370 can be performed in a different order. In these and other embodiments, any of the steps of the method 370 (e.g., block 373) can be performed before, during, and/or after any of the other steps of the method 370) (e.g., block 372) Moreover, a person of ordinary skill in the relevant art will recognize that the illustrated method 370) can be altered and still remain within these and other embodiments of the present technology. For example, one or more steps of the method 370) (e.g., block 373) illustrated in FIG. 3 can be omitted and/or repeated in some embodiments.

As one skilled in the art will appreciate, any of the screen assemblies and/or screens described above can be used as part of an adjustable shunting system, e.g., to control the flow of fluid therethrough. Moreover, certain features described with respect to one screen assembly and/or screen can be added or combined with another screen assembly and/or screen. Accordingly, the present technology is not limited to the screen assemblies and actuators expressly identified herein. For example, the screen assemblies could be utilized with the adjustable shunting systems and actuation assemblies described in U.S. Pat. Nos. 11,291,585 and 11,166,849, and International Patent Application Nos. PCT/US20/55144, PCT/US20/55141, PCT/US21/14774, PCT/US21/18601, PCT/US21/023238, and PCT/US21/27742, the disclosures of which were previously incorporated by reference herein in their entireties and for all purposes. Accordingly, although the screen assemblies are described in the context of specific adjustable shunting systems and actuation assemblies, screen assemblies configured in accordance with embodiments of the present technology can be used with any of the adjustable shunting systems, actuation assemblies, and/or actuators previously incorporated by reference. Likewise, although the screen assemblies are described in the context of adjustable shunting systems, the screen assemblies described herein can be used to filter fluid flow through other types of medical devices.

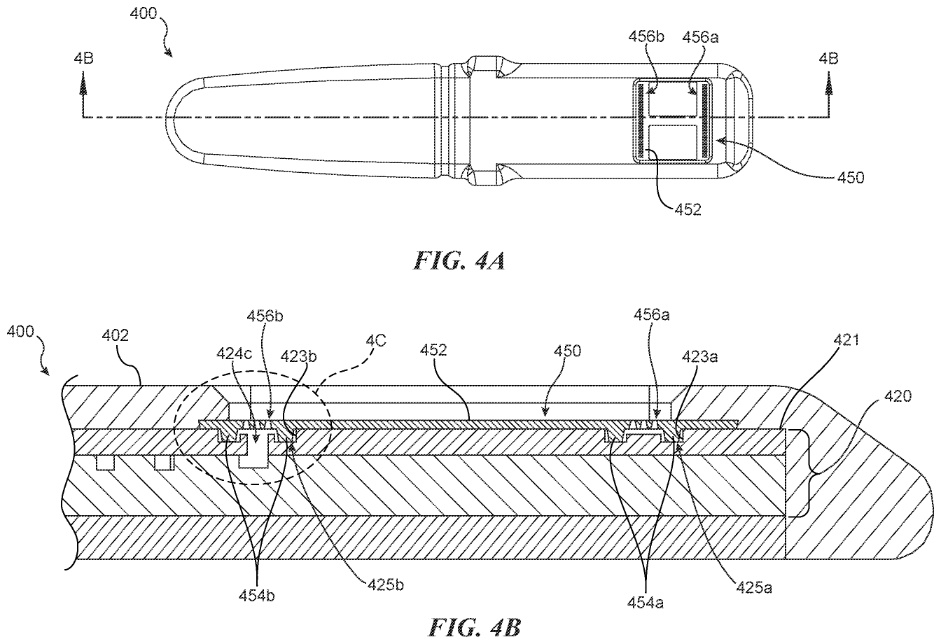

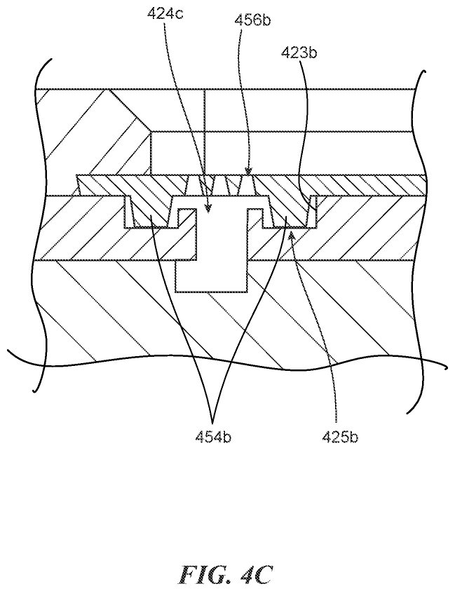

FIGS. 4A and 4B are top and cross-sectional views, respectively, of another adjustable shunting system 400 (“the system 400”) including a screen assembly 450 configured in accordance with various embodiments of the present technology. At least some aspects of the system 400 can be generally similar or identical in structure and/or function to one or more aspects of the system 100 of FIGS. 1A and 1B, with like names and/or reference numbers (e.g., plate assembly or cartridge 420 versus the plate assembly 120 of FIGS. 1A-2) indicating generally similar or identical aspects. Additionally, or alternatively, at least some aspects of the screen assembly 450) can be generally similar or identical in structure and/or function to one or more aspects of the screen assembly 150 of FIGS. 1A-2. In these and other embodiments, the system 400 can perform and/or be configured for use in one or more steps of the method 370 of FIG. 3.

Referring to FIGS. 4A and 4B together, the screen assembly 450 includes a filter or screen 452 having one or more screening or filtering elements 456 (shown as one or more first screen elements 456a and one or more second screen elements 456b in FIGS. 4A and 4B). As best seen in FIG. 4B, the screen assembly 450 can further include one or more sealing elements 454 (e.g., a first sealing element 454a and a second sealing element 454b). In the illustrated embodiment, both the first sealing element 454a and the second sealing element 454b are configured as projections extending (e.g., downwardly) from the screen 452. In other embodiments, however, one or both of the first sealing elements 454a and the second sealing element 454b can be separate components from the screen 452 and positioned between the screen 452 and the plate assembly 420. In these and other embodiments, one or both of the first sealing elements 454a and the second sealing element 454b can be formed from a same or different material, e.g., as each other, the screen 452, and/or another component of the system 400.

The sealing elements 454a, 454b can correspond to one or more of the screen elements 456a, 456b. In the illustrated embodiment, for example, the first sealing element 454a is positioned around (e.g., extends around, surrounds, encircles, borders, defines, and/or the like) a perimeter and/or circumference of the one or more first screen elements 456a and the second sealing element 454b is positioned around a perimeter and/or circumference of the one or more second screen elements 456b. In other embodiments, one or both of the first sealing element 454a and the second sealing element 454b can be positioned around a portion of the perimeters/circumferences of the respective one or more first and second screen elements 456a. 456b and/or extend partially or fully around individual ones of the respective first and second screen elements 456a, 456b.

Additionally, the sealing elements 454a, 454b can sealingly engage the plate assembly 420 and create substantially fluid-impermeable seals (e.g., a first seal 425a and a second seal 425b) between the screen 452 and the plate assembly 420, such that all or substantially all fluid that passes through (e.g., is filtered by) the filtering elements 456a, 456b enters the plate assembly 420. In the illustrated embodiment, for example, the first sealing element 454a and the second sealing element 454b both sealingly engage a cover plate 421 of the plate assembly 420. The cover plate 421 can include one or more recessed areas (e.g., the first recessed area 423a and the second recessed area 423b) configured to correspond to individual ones of the sealing elements 454a, 454b. One or both of the first recessed area 423a and the second recessed area 423b can extend into and/or out of the cross-section plane shown in FIG. 4B through the cover plate 421, e.g., to define a channel extending through the cover plate 421 in a width-wise direction and/or at least generally perpendicular to a longitudinal axis of the system 400. Similarly, one or both of the sealing elements 454a, 454b can extend into and/or out of the cross-section plane shown in FIG. 4B along the screen 452, e.g., to define a perimeter and/or circumference around the corresponding screen elements 456a, 456b. Referring additionally to FIG. 4C, which is an enlarged view of region 4C in FIG. 4B, the second sealing element 454b can be received within the second recessed area 423b to form the second seal 425b; likewise, although not shown in FIG. 4C, the first sealing element 454a can be received within the first recessed area 423a to form the first seal 425a. In some aspects, sealingly engaging the sealing elements 454a, 454b within the corresponding recessed areas 423a, 423b is expected to further improve the substantially fluid-impermeable seal formed between the screen assembly 450 and the plate assembly 420.

The cover plate 421 can include one or more fluid inlets. Although only the third fluid inlet 424c is shown in FIG. 4B, it will be appreciated that the system 400 can also include a first fluid inlet and a second fluid inlet, such as the first and second fluid inlets 124a, 124b of FIG. 1C. One or more of the screen elements 456a, 456b can be aligned with (e.g., covering, positioned above, and the like) one or more of the fluid inlets, such that fluid can pass through the screen 452 before entering the plate assembly 420 via the fluid inlets (e.g., fluid first passes through the screen 452 before flowing through the fluid inlet 424c and into the plate assembly 420). In the illustrated embodiment, for example, the second screen elements 456b are aligned with the third fluid inlet 424c. Thus, when the sealing elements 454a, 454b sealingly engage the cover plate 421, the sealing elements 454a, 454b can also extend around the fluid inlets, such that the associated seals 425a, 425b are formed around the fluid inlets such that substantially all the fluid that flows through the screening elements 456a, 456b also flows into the plate assembly 420 via the fluid inlets. In the illustrated embodiment, for example, the second sealing element 454b extends around the third fluid inlet 424c such that the second seal 425b is formed around the third fluid inlet 424c and substantially all fluid that flows through the second screening elements 456b enters the plate assembly 420 via the third fluid inlet 424c. Accordingly, the sealing elements 454a, 454b can at least partially or fully prevent fluid that flows through the screen assembly 450 from leaking, for example, between the housing 402 and the plate assembly 420.

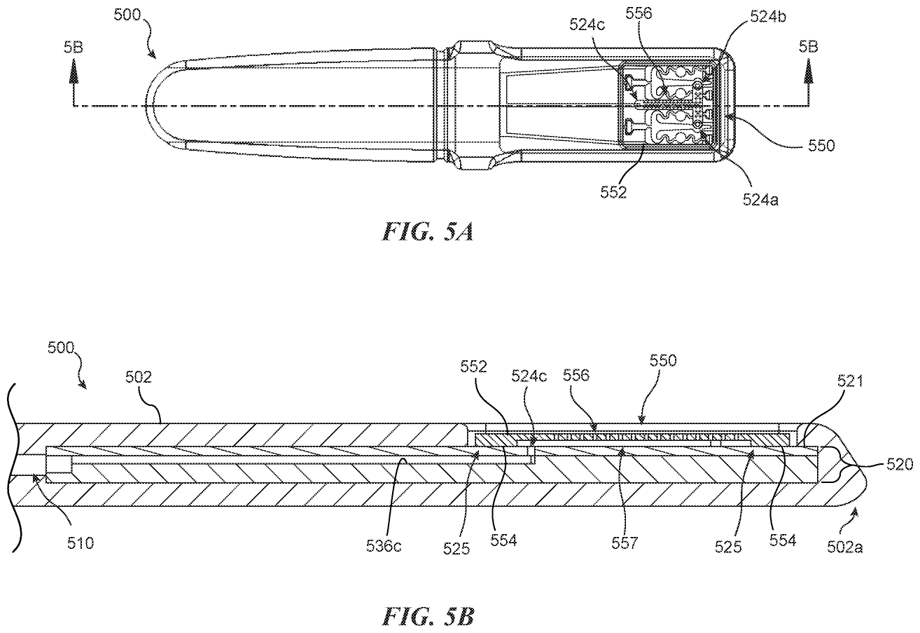

FIGS. 5A and 5B are top and cross-sectional views, respectively, of another adjustable shunting system 500 (“the system 500”) including a screen assembly 550 configured in accordance with various embodiments of the present technology. At least some aspects of the system 500 can be generally similar or identical in structure and/or function to one or more aspects of the system 100 of FIGS. 1A and 1B and/or the system 400 of FIGS. 4A and 4B, with like names and/or reference numbers (e.g., plate assembly or cartridge 520 versus the plate assembly 120 of FIGS. 1A-2 and the plate assembly 420 of FIGS. 4A and 4B) indicating generally similar or identical aspects. Additionally, or alternatively, at least some aspects of the screen assembly 550) can be generally similar or identical in structure and/or function to one or more aspects of the screen assembly 150 of FIGS. 1A-2 and/or the screen assembly 450 of FIGS. 4A and 4B. In these and other embodiments, the system 500 can perform and/or be configured for use in one or more steps of the method 370 of FIG. 3.

Referring to FIG. 5A, the system 500 includes a first fluid inlet 524a, a second fluid inlet 524b and a third fluid inlet 524c. The inlets 524a-c can be formed in a cover plate 521 (FIG. 5B) of the plate assembly 520 (FIG. 5B). In some embodiments, the cover plate 521 and/or the plate assembly 520 can be angled (e.g., radially inwardly) relative to a longitudinal axis of the system 500. The screen assembly 550 includes a screen 552 aligned with (e.g., positioned above) each of the first, second, and third fluid inlets 524a-c. The screen 552 includes one or more screen elements 556. In the illustrated embodiment, screen elements 556 extend at least partially between the first, second, and/or third fluid inlets 524a-c to form a T-shaped array of screen elements. In other embodiments, the screen elements 556 can be arranged in one or more rows, columns, a U-shaped array, a V-shaped array, and/or any other suitable configuration.

Referring to FIG. 5B, the screen 552 can include one or more sealing elements 554 configured to sealingly engage the plate assembly 520 and form a substantially fluid-impermeable seal 525 between the screen assembly 550 and the plate assembly 520. In the illustrated embodiment, for example, the screen assembly 550 includes a single sealing element 554 configured to form a single seal 525 extending around the screen elements 556. In other embodiments, however, the screen assembly 550) can include more sealing elements 554, individual ones of which can be configured to form respective seals 525 with the plate assembly 520. In these and other embodiments, the sealing elements 554 can extend or project from screen 552, as described above with respect to the sealing elements 454a, 454b of FIGS. 4A and 4B. Accordingly, as best seen in FIG. 5B, sealingly engaging the screen assembly 550 with the plate assembly 520 can create a fluid space or gap 557 between the sealing elements 554, the plate assembly 520, and the screen 552, as shown in FIG. 5B. The fluid space 557 can be fluidly coupled to the screen elements 556 and the fluid inlets 524a-c (FIG. 5A) in the plate assembly 520) and can allow fluid to flow between individual ones of the fluid inlets 524a-c within the housing 502 upstream from the plate assembly 520. Accordingly, fluid that flows through the screen elements 556 can pass through the fluid space 557 before entering the plate assembly 520 via the fluid inlets 524a-c and flowing through the channels (only third channel 536c is visible in FIG. 5B) to the system's main fluid conduit 510. If one or more of the screen elements 556 proximate a given fluid inlet (e.g., the third fluid inlet 524c) is obstructed, the fluid space 557 can allow fluid that enters the system 500 to reach the given fluid inlet via one or more of the other, unobstructed screen elements 556 away from the given fluid inlet. Accordingly, the fluid space 557 is expected to maintain flow through the system 500 and/or improve the control of flow through the system 500 if individual ones of the screen elements 556 become blocked or clogged.

The plate assembly 520 can be sloped or tapered. In at least some embodiments, for example, the plate assembly 520 has a first dimension (e.g., height) at or near the first end portion 502a and a second dimension distal from the first end portion 502a (e.g., at or near the main fluid conduit 510) that is less than the first dimension, such that the plate assembly 520 is sloped/angled downwardly toward the main fluid conduit 510. Additionally, or alternatively, one or more of the channels 536 can be angled or sloped. In the illustrated embodiment, for example, the third channel 536c is angled relative to the longitudinal axis of the system 500, e.g., inwardly from the screen assembly 550 toward the main fluid conduit 510 and/or the interior of the housing 502. In some aspects, angled plate assemblies and/or channels are expected to reduce the likelihood that fluid will accumulate within the fluid space 557 and at least partially block flow through the system 500 during operation.

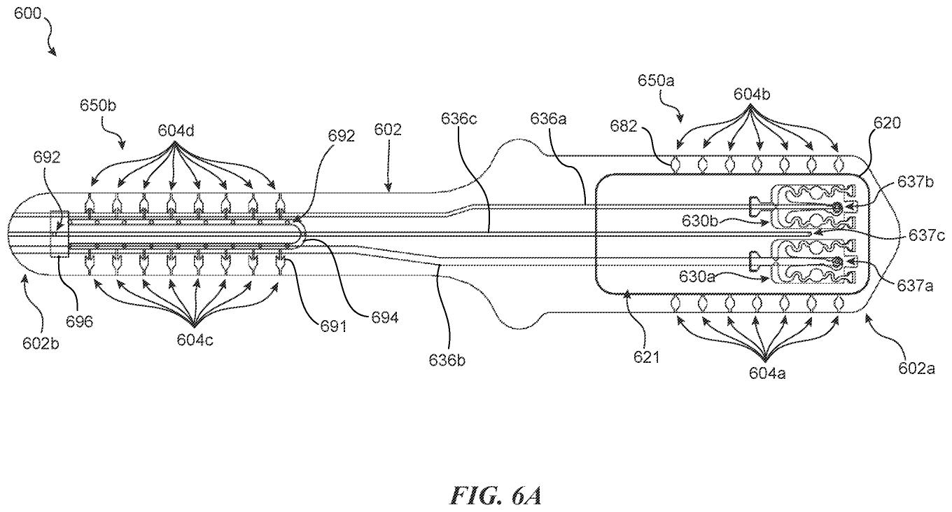

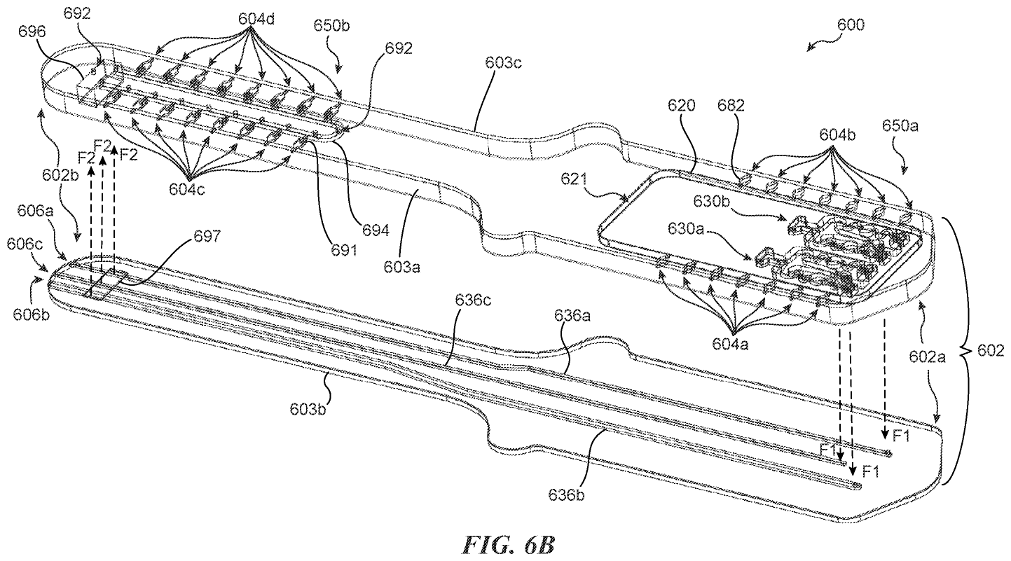

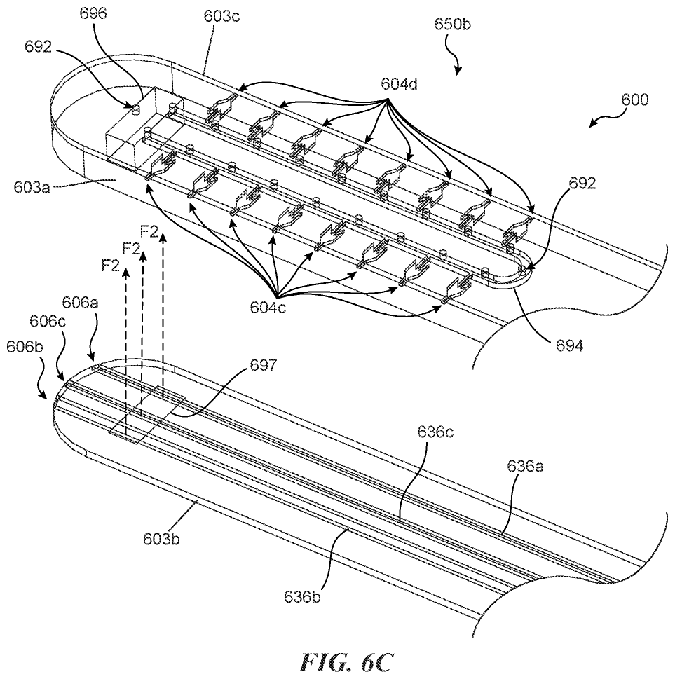

FIG. 6A is a top view, and FIGS. 6B and 6C are partially-exploded isometric views of another adjustable shunting system 600 (“the system 600”) including screen assemblies 650a, 650b configured in accordance with various embodiments of the present technology. At least some aspects of the system 600 can be generally similar or identical in structure and/or function to one or more aspects of the system 100 of FIGS. 1A and 1B, the system 400 of FIGS. 4A and 4B, and/or the system 500 of FIGS. 5A and 5B, with like names and/or reference numbers (e.g., cartridge 620 versus the plate assembly 120 of FIGS. 1A-2, the plate assembly 420 of FIGS. 4A and 4B, and the plate assembly 520 of FIGS. 5A and 5B) indicating generally similar or identical aspects. In these and other embodiments, the system 600 can perform and/or be configured for use in one or more steps of the method 370 of FIG. 3.

Referring to FIGS. 6A and 6B together, the system 600 includes a generally elongated housing 602 and a plate assembly or cartridge 620. The housing 602 extends between a first end portion 602a and a second end portion 602b. The system 600 can include one or more openings or ports 604a-d (e.g., inlets and/or outlets) positioned within the housing 602 and through which fluid (e.g., aqueous) can enter and/or exit the interior of the system 600. In the illustrated embodiment, for example, the system 600 includes one or more first ports 604a, one or more second ports 604b, one or more third ports 604c, and one or more fourth ports 604d (referred to collectively as “ports 604”). In the illustrated embodiment, the first and second ports 604a, 604b are fluidly coupled to the cartridge 620 and positioned in or near the first end portion of the housing 602, with the first ports 604a positioned on a first side of the cartridge 620, and the second ports 604b positioned on a second side of the cartridge 620 opposite the first side. Additionally, in the illustrated embodiment, the third and fourth ports 604c, 604d are spaced apart from the cartridge 620 in or near the second end portion 602b of the housing 602, with the third ports 604c positioned on a same side of the housing 602 as the first ports 604a and the fourth ports 604d positioned on a same side of the housing 602 as the second ports 604b. In other embodiments, individual ones of the ports 604a-d can have another suitable configuration. In some aspects, having multiple ports 604a-d is expected to improve the clog-resistance of the system 600 and/or improve the ability of the system 600 to control fluid flow if one or more of the ports 604a-d become clogged or have otherwise reduced flow.

Individual ones of the first and second ports 604a, 604b can be fluidly coupled to a chamber 621 within the housing 602 configured to receive fluid (e.g., aqueous) therefrom. The cartridge 620 is positioned at least partially or fully within the housing 602 and configured to control the flow of fluid that enters the system 600, e.g., via individual ones of the ports 604a-d. For example, the illustrated cartridge 620 is configured to control the flow of fluid that enters the chamber 621 via individual ones of the ports 604a, 604b. The cartridge 620 can include: (i) one or more channels 636a-c through which the fluid within the chamber 621 can flow: (ii) one or more channel inlets 637a-c that fluidly couple individual ones of the channels 636a-c to the chamber 621; and (iii) one or more actuators 630a-b positioned to control the flow of fluid (shown using dashed line arrows F1 in FIG. 6B) through a corresponding one of the channel inlets 637a-c. For illustration purposes, the channel inlets 637a-c are not labeled in FIG. 6B. The individual channel inlets 637a-c, the corresponding channels 636a-c, and/or the associated outlets 606a-c (FIG. 6B) can be discrete features and spaced apart from one another. Accordingly, fluid that enters the chamber 621 via individual ones of the ports 604a, 604b can flow through individual ones of the channel inlets 637a-c (e.g., depending on the actuated state of the corresponding actuator 630a-b), into the corresponding channel 636a-c, and flow out of the system 600 via the associated outlet 606a-c. The individual outlets 606a-c can be positioned at the second end portion 602b of the housing, e.g., at a distal end of the system 600 and/or opposite the ports 604a-b, with the individual outlets 606a-c spaced apart from each other. Individual ones of the first and/or second ports 604a, 604b can be configured to reduce or prevent formation of blockages that affect rate of flow through the system 600 and/or reduce or prevent changes to resistance to flow through the system 600 when one or more of the ports 604a-b are blocked, such that flow through the system 600 is expected to be maintained without or substantially without interference from blockages. As best seen in FIG. 6A, in the illustrated embodiment, each of the first and second ports 604a, 604b include a widened portion 682 configured to reduce or prevent tissue growth into individual ones of the first and second ports 604a, 604b.

Referring to FIGS. 6A and 6B together, in the illustrated embodiment, the third channel inlet 637c is configured to remain in an open state, such that fluid within the chamber 621 can flow through the third channel inlet 637c, into the third channel 636c, and/or exit the system 600 via the third outlet 606a without or substantially without interference from one or more of the actuators 630. Additionally, or alternatively, one or both of the first and second channel inlets 637a-b can be configured to (i) allow fluid flow therethrough at a first flow rate when the corresponding actuator 630a-b is in the closed position, and (ii) allow fluid flow therethrough at a second flow rate greater than the first flow rate when the corresponding actuator 630a-b is in the open position, such that actuating the actuators 630a-b can change a resistance to flow through the channel inlets 637a-b, but some flow through the channel inlets 637a-b is maintained independent of an actuated state of the actuators 630a-b. In some embodiments, maintaining flow through the channel inlets 637a-c and/or the corresponding channels 636a-c is expected to reduce or prevent fluid stagnation within the channel inlets 637a-c and/or the channels 636a-c, reduce or prevent formation of blockages that affect flow through the system 600, and/or otherwise improve patency to fluid flow of the system 600 during operation.

FIG. 6C is an enlarged view of the screen assembly 650b of FIG. 6B with other portions of the system 600 omitted for purposes of illustration/clarity. Referring to FIGS. 6B and 6C together, individual ones of the third and fourth ports 604c, 604d can be fluidly coupled to one or more of the channels 636a-c. Each of the third and fourth ports 604c, 604d can be coupled to the channels 636a-c via an outlet channel 694 and an internal reservoir 696. The outlet channel 694 can be fluidly coupled to individual ones of the third and/or fourth ports 604c, 604d. The internal reservoir 696 can be downstream from the outlet channel 694, between the outlet channel 694 and the channels 636a-c, and fluidly couple the outlet channel 694 to individual ones of the channels 636a-c.

During operation, fluid (e.g., aqueous) can enter the internal reservoir 696 via one or more of the channels 636a-c, such as when one or more outlets 606 (individually identified as a first outlet 606a of the first channel 636a, a second outlet 606b of the second channel 636b, and a third outlet of the third channel 636c in FIGS. 6B and 6C) are partially or fully blocked/obstructed. The internal reservoir 696 can be fluidly coupled to one or more of the channels 636a-c at interface region 697, such that fluid flowing through the channels 636a-c can enter and/or begin to fill the internal reservoir 696, generally flowing in the direction indicated by dashed-line arrows F2 in FIG. 6C. As the fluid fills the internal reservoir 696, the fluid can flow from the internal reservoir 696 into the outlet channel 694 and flow out of the system 600 via individual ones of the third and fourth ports 604c, 604d. Additionally, or alternatively, the outlet channel 694 and/or the internal reservoir 696 can include one or more additional outlets 692, which may or may not be configured to reduce or prevent blockages, and through with fluid can flow out of the system 600. In these and other embodiments, individual ones of the channels 636a-c can be configured to allow fluid to exit the system 600 through the second end portion 602b directly via the channels 636a-c.

Additionally, individual ones of the third and fourth ports 604c, 604d can be configured to reduce or prevent blockages that affect rate of flow through the system 600 and/or reduce or prevent changes to resistance to flow through the system 600 when one or more of the ports 604a-b are blocked, such that flow through the system 600 is expected to be maintained without or substantially without interference from blockages. For example, as best seen in FIG. 6A, in the illustrated embodiment each of the third and fourth ports 604c, 604d include an inwardly-angled portion 691 configured to reduce or prevent tissue growth into individual ones of the third and fourth ports 604c, 604d.

As best seen in FIGS. 6B and 6C, the housing 602 can comprise one or more housing portions or layers 603 (individually identified as a first or intermediate layer 603a, a second or lower layer 603b, and a third or upper layer 603c in FIGS. 6B and 6C). Each of the layers 603 can be flexible and/or formed from an elastomeric material, such as silicone. The thickness of one or more of the layers 603 can be less than, equal to, or greater than the thickness of one or more of the other layers 603, such that each layer 603 can have a same or different thickness as one or more of the other layers 603. In some embodiments, each of the layers 603 are formed separately and assembled together to form the system 600. For example, the second layer 603b can be positioned on a first side of the first layer 603a and the third layer 603c can be positioned on a second side of the first layer 603a opposite the first side. Individual ones of the layers 603a-c can be separate structures coupled to one another to form the system 600 and/or respective regions of a continuous structure forming the system 600. Individual ones of the ports 604a-d, the chamber 621, the channel inlets 637a-c, the channels 636a-c, the outlets 606, the fluid reservoir 696, the outlets 692, and/or the outlet channel 694 can be positioned within and/or defined by one or more of the layers 603a-c. In the illustrated embodiment, for example, the first layer 603a includes the ports 604a-d, the chamber 621, the channel inlets 637a-c, the fluid reservoir 696, and the outlet channel 694, the second layer 603b includes the channels 636a-c and the outlets 606a-c, and the third layer 603c includes the outlets 692.

During operation of the system 600, fluid can enter the system 600 via one or more of the layers 603a-c, flow through the system 600 (e.g., horizontally and/or longitudinally) within one or more of the layers 603a-c, flow between (e.g., vertically) one or more of the layers 603a-c, and/or flow out of the system 600 via one or more of the layers 603a-c. In the illustrated embodiment, for example, fluid can enter the first layer 603a via one or more of the ports 604a-b, flow from the first layer 603a toward and/or into the second layer 603b via one or more of the channel inlets 637a-c (FIG. 6A) and flow through/within the second layer 603b via one or more of the channels 636a-c. Fluid within the second layer 603b (e.g., within one or more of the channels 636a-c) can exit the second layer 603b via one or more of the outlets 606a-c and/or return to the first layer 603a via the fluid reservoir 696. Fluid within the fluid reservoir can flow through the first layer 603a via the outlet channel 694 and exit the system 600 via one or more of the ports 604c-d in the first layer 603a and/or one or more of the outlets 692 in the third layer 603c.

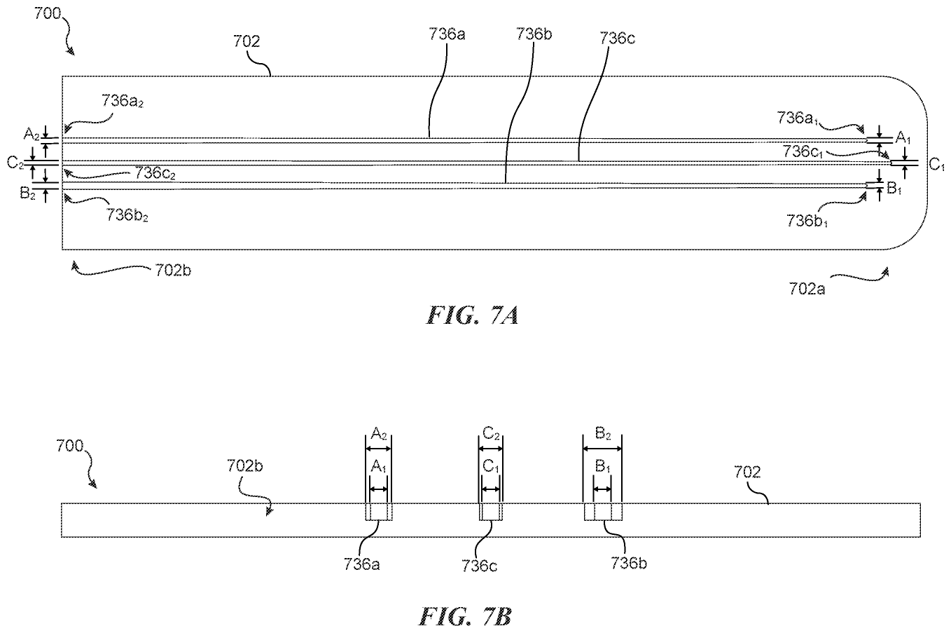

FIGS. 7A and 7B are a top view and an end view, respectively, of select aspects of an adjustable shunting system 700 (“system 700”) configured in accordance with embodiments of the present technology. Other aspects of the system 700 of FIGS. 7A and 7B are omitted for illustrative clarity. At least some aspects of the system 700 can be generally similar or identical in structure and/or function to one or more aspects of the system 100 of FIGS. 1A and 1B, the system 400 of FIGS. 4A and 4B, the system 500 of FIGS. 5A and 5B, and/or the system 600 of FIGS. 6A-6C, with like names and/or reference numbers (e.g., first end portion 702a versus the first end portion 102a of FIGS. 1A and 1B) indicating generally similar or identical aspects. In these and other embodiments, the system 700 can perform and/or be configured for use in one or more steps of the method 370 of FIG. 3.

The system 700 can include a housing 702 having a first end portion 702a and a second end portion 702b. The system 700 further includes one or more channels 736a-c, individual ones of which can extend at least partially between the first end portion 702a and the second end portion 702b of the housing 702. In the illustrated embodiment, the system 700 includes a first channel 736a, a second channel 736b, and a third channel 736c. In other embodiments, however, the system 700 can include more or fewer flow channels 736. Individual ones of the channels 736a-c can have a respective first end 736a₁, 736b₁, 736c₁ at or near the first end portion 702a, a respective second end 736a₂, 736b₂, 736c₂ at or near the second end portion 702b and/or opposite the respective first end 736a₁, 736b₁, 736c₁, and one or more dimensions that vary between the respective first and second ends 736a₁₋₂, 736b₁₋₂, 736c₁₋₂. The first and second ends 736a₁₋₂, 736b₁₋₂, 736c₁₋₂ are not labeled in FIG. 7B.

In the illustrated embodiment, for example, a width of each of the channels 736a-c increases from a respective first width A1, B1, C1 at the first end 736a₁, 736b₁, 736c₁ to a respective second width A2, B2, C2 at the second end 736a₂, 736b₂, 736c₂. In these and other embodiments, the height, diameter, cross-sectional area, and/or another suitable dimension of one or more of the channels 736a-c can vary from a first value at the first end 736a₁, 736b₁, 736c₁ to a second value at the second end 736a₂, 736b₂, 736c₂ that is different (e.g., greater) than the first value. In the illustrated embodiment, the dimension(s) of the channels 736a-c change along the entire length of individual ones of the channels 736a-c. In other embodiments, the dimension(s) of individual ones of the channels 736a-c can change along a portion of the respective channel, such that individual ones of the channels 736a-c can have one or more constant dimensions along a first portion of the respective channel 736a-c and one or more varying dimensions along a second portion of the respective channel 736a-c.

In some aspects, channels having one or more varying dimensions are expected to improve the flow of fluid through adjustable shunting systems. Fluid (e.g., aqueous) can flow through individual ones of the channels 736a-c from the first end portion 702a toward the second end portion 702b and exit the system 700. As the fluid flows through the channels 736a-c toward the second end portion 702b, the increased dimension(s) of the channels 736a-c are expected to reduce or prevent blockages or clogs within the channels 736a-c that may affect fluid flow through the system 700.

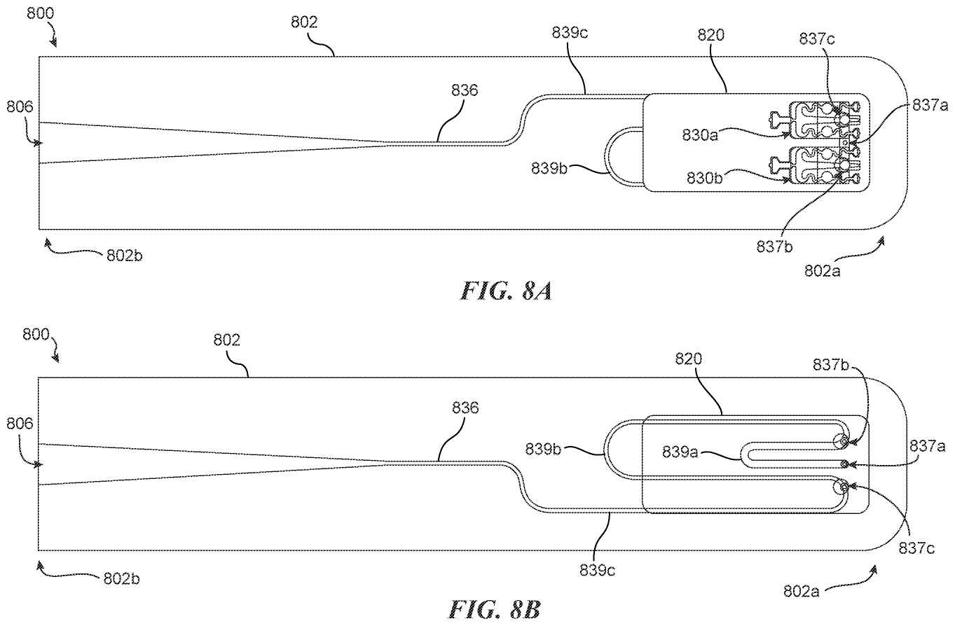

FIGS. 8A and 8B are a top view and a bottom view, respectively, of an adjustable shunting system 800 (“system 800”) configured in accordance with embodiments of the present technology. At least some aspects of the system 800 can be generally similar or identical in structure and/or function to one or more aspects of the system 100 of FIGS. 1A and 1B, the system 400 of FIGS. 4A and 4B, the system 500 of FIGS. 5A and 5B, the system 600 of FIGS. 6A-6C, and/or the system 700 of FIGS. 7A and 7B, with like names and/or reference numbers (e.g., plate assembly or cartridge 820 versus the plate assembly 120 of FIGS. 1A-2, the plate assembly 420 of FIGS. 4A and 4B, the plate assembly 520 of FIGS. 5A and 5B, and the cartridge 620 of FIGS. 6A-6C) indicating generally similar or identical aspects. In these and other embodiments, the system 800 can perform and/or be configured for use in one or more steps of the method 370) of FIG. 3.

The system 800 includes a housing 802 having a first end portion 802a and a second end portion 802b, a plate assembly 820, one or more fluid outlets 806, and one or more channels 836 (“channel 836”) that fluidly couple the plate assembly 820 to individual ones of the fluid outlets 806. The plate assembly 820 can include one or more actuators 830a-b. The channel 836 can be fluidly coupled to one or more inlets 837a-c and configured to receive fluid therefrom. Fluid flow through individual ones of the inlets 837a-c can be independently and/or selectively controlled by a corresponding one of the actuators 830a-b. In the illustrated embodiment, for example, the channel 836 includes a first inlet 837a open to fluid flow (e.g., always open to fluid flow and/or not configured to be selectively opened/closed by an actuator), a second inlet 837b configured to be selectively opened and/or closed to fluid flow by a first actuator 830a, and a third inlet 837c configured to be selectively opened and/or closed to fluid flow by a second actuator 830b. Individual ones of the inlets 837a-c can be positioned along the channel 836, such that the channel 836 can include multiple inlets 837a-c for receiving fluid and one or more channel portions or segments 839a-c defined by the inlets 837a-c. Individual ones of the inlets 837a-c can be positioned upstream and/or downstream from one or more of the other inlets 837a-c (e.g., the first inlet 837a is upstream from the second and third inlets 837b, 837c). Accordingly, the actuators 830a-b can be configured to control fluid flow into the channel 836 at various points (e.g., the inlets 837a-c) along the length of the channel 836. In the illustrated embodiment, the channel 836 begins at the first inlet 837a, includes the second inlet 837b downstream from the first inlet 837a, and includes the third inlet 837c downstream from the second inlet, such that the first, second, and third inlets 837a-c are in series along the length of the channel 836. With continued reference to the illustrated embodiment, the channel 836 includes a first channel segment 839a (best seen in FIG. 8B) between the first inlet 837a and the second inlet 837b, a second channel segment 839b between the second inlet 837b and the third inlet 837c, and a third channel segment 839c between the third inlet 837c and the outlet 806. In other embodiments, the system 800 can include more channels, and/or more or fewer channel segments, inlets, and/or actuators.

The position of each of the inlets 837a-c along the length of the channel 836 can be associated with a resistance to fluid flow through the channel 836. In the illustrated embodiment, for example, because the third inlet 837c is positioned downstream from the first inlet 837a, fluid entering the channel 836 via the third inlet 837c is expected to flow through a reduced length of the channel 836 relative to fluid entering the channel 836 via the first inlet 837a, such that the channel 836 provides less resistance to fluid that enters via the third inlet 837c as compared to the first inlet 837a. As another example, because the second inlet 837b is positioned upstream from the third inlet 837c and downstream from the first inlet 837a, fluid that enters the channel 836 via the second inlet 837b faces increased resistance to flow as compared to fluid that enters the channel 836 via the third inlet 837c but reduced resistance to flow as compared to fluid that enters the channel 836 via the first inlet 837a.

In some aspects, channels that include multiple inlets are expected to be less likely to become blocked or clogged, and/or can allow fluid to drain when portions of these channels become blocked or clogged. In at least some embodiments, for example, individual ones of the inlets 837a-c can be opened in response to an upstream channel blockage to “tap in” or allow fluid flow into the channel 836 at a location downstream from the upstream channel blockage. With reference to the illustrated embodiment, if the first channel segment 839a becomes blocked or clogged, the first actuator 830a can be actuated to allow fluid to enter the channel 836 via the second inlet 837b, bypassing the first channel segment 839a and the blockage/clog therein. With continued reference to the illustrated embodiment, if the second channel segment 839b becomes blocked or clogged, the second actuator 830b can be actuated to allow fluid to enter the channel 836 via the third inlet 837c, bypassing the second channel segment 839b and the blockage/clog therein. Additionally, or alternatively, actuating one or both of the actuators 830a-b to allow fluid flow through one or both of the second and third inlets 837b-c can change (e.g., decrease) a resistance to fluid flow through the system 800, as described above. Fluid can preferentially enter the channel 836 via the most downstream open inlet (e.g., all or substantially all the fluid can enter the channel 836 via the third inlet 837c when the third inlet 837c is open). In these and other embodiments, one or more of the inlets 837a-c, the channel 836 and/or one or more of the channel segments 839a-c thereof can include one or more varying dimensions, as described in detail with reference to FIGS. 7A and 7B. In the illustrated embodiment, for example, a width of the third channel segment 839c increases over a portion of the third channel segment's length, which can further reduce or prevent the likelihood of the channel 836 becoming blocked or clogged. In these and other, the varying dimensions of individual ones of the inlets 837a-c and/or the channel segments 839a-c are expected to improve the control of flow through the system 800, e.g., by providing multiple resistances to flow and/or, under a given pressure, multiple flow rates through the system 800 in response to allowing or prevent flow through individual ones of the inlets 837a-c.

### Examples

Several aspects of the present technology are set forth in the following examples:

1. A system for shunting fluid from a first body region to a second body region within a patient, the system comprising:
a first layer including a first fluid inlet and a second fluid inlet, each configured to receive fluid from the first body region; and
a second layer coupled to the first layer and including a first fluid outlet and a second fluid outlet, each configured to be positioned within the second body region, wherein the first fluid outlet is fluidly coupled to the first fluid inlet via a first channel and the second fluid outlet is fluidly coupled to the second fluid inlet via a second channel; and
an actuator positioned to control the flow of fluid from the first fluid inlet in the first layer into the first channel of the second layer,
wherein, independent of a state of the actuator, the second channel is configured to receive fluid from the second fluid inlet.

2. The system of example 1 wherein the first fluid outlet and the second fluid outlet are positioned at a distal end of the second layer, and wherein the first fluid outlet is spaced apart and discrete from the second fluid outlet.

3. The system of example 1 or example 2 wherein the first layer defines a chamber configured to receive fluid from the first body region, wherein the actuator is position within the chamber and configured to adjust the flow of fluid within the chamber through the first fluid inlet.

4. The system of any of examples 1-3 wherein the first layer includes a third fluid outlet fluidly coupled to one or both of the first channel and the second channel and fluid reservoir positioned downstream from the first and second fluid inlets and upstream from the third fluid outlet, wherein the fluid reservoir is configured to substantially prevent fluid flow through the third fluid outlet until the fluid reservoir is at least partially filled with fluid received from one or both of the first channel and the second channel.

5. The system of example 4 wherein the third fluid outlet is fluidly coupled to the fluid reservoir by a third channel extending between the fluid reservoir and the third fluid outlet.

6. The system of any of examples 1-5, further comprising a third layer including a third fluid outlet fluidly coupled to one or both of the first channel and the second channel.

7. The system of example 6 wherein the first layer includes a first side and a second side opposite the first side, wherein the second layer is coupled to the first side, and wherein the third layer is coupled to the second side.

8. The system of example 6 or example 7 wherein the first layer, the second layer, and the third layer are configured so that fluid flows (i) from the first layer to the second layer in a first direction perpendicular to a longitudinal axis of the system and (ii) from the second layer through the first layer to the third layer in a second direction opposite the first direction and perpendicular to the longitudinal axis.

9 The system of any of examples 6-8 wherein first layer includes a fluid reservoir configured to fluidly couple one or both of the first channel and the second channel to the third fluid outlet.

10. The system of example 9 wherein the first layer includes a third channel configured to fluidly couple the fluid reservoir and the third fluid outlet.

11. The system of example 10 wherein at least a first portion of one or both of the first channel and the second channel are configured to direct fluid flow in a first direction, and wherein at least a second portion of the third channel is configured to direct fluid flow in a second direction opposite the first direction.

12. The system of any of examples 1-11 wherein, independent of a state of the actuator, the first channel is configured to receive fluid from the first fluid inlet

13. The system of any of examples 1-12 wherein the actuator is configured to transition between (i) a first position in which the actuator allows fluid to flow between the first fluid inlet and the first fluid outlet at a first rate and (ii) a second position in which the actuator allows fluid to flow between the first fluid inlet and the first fluid outlet at a second rate less than the first rate.

14. A system for shunting fluid from a first body region to a second body region within a patient, the system comprising:
a housing at least partially defining a channel configured to allow fluid flow from the first body region to the second body region, wherein the channel includes—
   a first inlet, and
   a second inlet positioned downstream from the first inlet; and
an actuator positioned to control the flow of fluid through the second inlet, wherein the actuator is configured to transition between a first position and a second position,
wherein—
   when the actuator is in the first position, the channel provides a first resistance to fluid flow therethrough, and
   when the actuator is in the second position, the channel provides a second resistance to fluid flow therethrough, the second resistance greater than the first resistance.

15. The system of example 14 wherein, when the actuator is in the first position, at least a portion of the actuator is offset from second inlet, and wherein, when the actuator is in the second position, the portion of the actuator is at least partially aligned with the second inlet.

16. The system of example 14 or example 15 wherein, in the first position, the actuator allows fluid to flow through the second inlet at a first rate, and wherein, in the second position, the actuator allows fluid to flow through the second inlet at a second rate less than the first rate.

17. The system of any of examples 14-16 wherein, independent of a position of the actuator, the first inlet is configured to allow fluid to flow into the channel.

18. The system of any of examples 14-17 wherein, when the actuator is in the first position, substantially all fluid flow into the channel is via the second inlet.

19. The system of any of examples 14-18 wherein the housing comprises a first layer and a second layer coupled to the first layer.

20. The system of example 19 wherein the first layer at least partially defines the channel, and wherein the second layer at least partially defines a chamber configured to receive the actuator.

21. A system for shunting fluid from a first body region to a second body region within a patient, the system comprising:
a housing including a plurality of fluid inlets, wherein individual ones of the plurality of fluid inlets include a widened middle portion;
a plate assembly within the housing, the plate assembly including—
   a chamber fluidly coupled to at least one of the fluid inlets, and
an actuator positioned within the chamber and configured to selectively control the flow of fluid through the system.

22. The system of example 21 wherein the plurality of fluid inlets is a plurality of first fluid inlets, the system further comprising a plurality of second inlets, wherein individual ones of the plurality of second inlets include an angled middle portion.

23. The system of example 22, further comprising a channel fluidly coupled to the plate assembly, wherein the actuator is positioned to selectively control the flow of fluid through the channel, and wherein individual ones of the plurality of second inlets are fluidly coupled to the channel.

24. The system of example 23, further comprising a fluid reservoir, wherein the fluid reservoir fluidly couples individual ones of the plurality of second inlets to the channel.

25. The system of example 23 or example 24 wherein the channel has a first end and a second end opposite the first end, and wherein the first end of the channel has a first dimension, and the second end of the channel has a second dimension different than the first end.

26. The system of example 25 wherein the first dimension is a first width, wherein the second dimension is a second width and the second width is greater than the first width.

27. The system of example 25 wherein the first dimension is a first cross-sectional area and the second dimension is a second cross-sectional area, and wherein the second cross-sectional area is greater than the first cross-sectional area.

28. A system for shunting fluid from a first body region to a second body region within a patient, the system comprising:
a housing;
a plate assembly within the housing, the plate assembly including—
   a chamber,
   a plurality of fluid inlets positioned within the chamber,
   a flow channel, wherein each of the plurality of fluid inlets are fluidly coupled to the flow channel, and
   an actuator positioned within the chamber and configured to control the flow of fluid through the flow channel.

29. The system of example 28 wherein each of the plurality of fluid inlets are fluidly coupled to the channel in series.

30. The system of example 28 or example 29 wherein the plurality of fluid inlets include a first fluid inlet and a second fluid inlet, and wherein the second fluid inlet is positioned downstream from the first fluid inlet, and further wherein the actuator is configured to control the flow of fluid the second fluid inlet.

31. The system of any of examples 28-30 wherein the flow channel has a first end and a second end opposite the first end, and wherein the first end of the flow channel has a first dimension and the second end of the flow channel has a second dimension different than the first end.

32. The system of example 31 wherein the first dimension is a first width and the second dimension is a second width, and wherein the second width is greater than the first width.

33. The system of example 31 wherein the first dimension is a first cross-sectional area and the second dimension is a second cross-sectional area, and wherein the second cross-sectional area is greater than the first cross-sectional area.

34. A screen assembly for use with an adjustable shunting system for treating a patient, the screen assembly comprising:
a screen having a first end portion and a second end portion spaced apart from the first end portion,
wherein the first end portion of the screen is at least partially aligned with fluid inlets of the shunting system, and
wherein, during operation, the screen is configured to (i) at least partially prevent debris from entering the fluid inlets of the shunting system, and (ii) receive non-invasive ablative energy at the first end portion to at least partially remove the debris from the screen.

35. The screen assembly of example 34 wherein the screen further includes one or more actuator access regions positioned at least partially between the first end portion of the screen and the second end portion of the screen.

36. The screen assembly of example 35 wherein each of the actuator access regions is at least partially aligned with at least one actuator of the shunting system.

37. The screen assembly of example 35 or example 36 wherein each of the one or more actuator access regions is (i) an aperture formed in the screen or (ii) at least partially transparent.

38. The screen assembly of any of examples 34-37 wherein:
the first end portion includes a plurality of screening elements, and
each of the fluid inlets is at least partially aligned with one or more of the plurality of screening elements.

39. The screen assembly of example 38 wherein each of the plurality of screening elements includes a pore formed in the screen, the individual pores being configured to at least partially prevent debris from entering the fluid inlets of the shunting system.

40. The screen assembly of example 38 or example 39 wherein each of the plurality of screening elements has a width between 0.1 μm and 100 μm.

41. The screen assembly of any of examples 38-40 wherein each of the plurality of screening elements has a width of 10 μm.

42. The screen assembly of any of examples 38-41 wherein each of the plurality of screening elements has a circular, oval, square, pentagonal, hexagonal, curvilinear, or rectilinear shape.

43. The screen assembly of any of examples 38-42 wherein the plurality of screening elements is a plurality of first screening elements and the plurality of fluid inlets is a plurality of first fluid inlets, wherein:
the second end portion of the screen is at least partially aligned with one or more second fluid inlets of the shunting system;
the second end portion further includes a plurality of second screening elements; and
each of the second fluid inlets is at least partially aligned with one or more of the plurality of second screening elements.

44. The screen assembly of example 43 wherein each of the plurality of second screening elements includes a pore formed in the second end portion of the screen.

45. The screen assembly of example 43 or example 44 wherein the first screening elements and the second screening elements have the same dimension.

46. The screen assembly of any of examples 43-45 wherein the first screening elements and the second screening elements have the same shape.

47. The screen assembly of any of examples 34-46 wherein the screen is formed at least partially from polydimethylsiloxane (PDMS), polydimethylacrylamide (PDMA), or super-elastic nitinol.

48. The screen assembly of any of examples 34-47, further comprising a sealing element configured to sealingly engage with the adjustable shunting system to form a substantially fluid-impermeable seal therewith.

49. The screen assembly of example 48 wherein the sealing element is configured to extend at least partially around the fluid inlets.

50. The screen assembly of example 48 or example 49 wherein the sealing element extends outwardly away from the screen.

51. The screen assembly of any of examples 34-50 wherein the screen defines a fluid space between the screen and the fluid inlets.

52. The screen assembly of example 51 wherein the screen includes a plurality of screening elements, and wherein the fluid space fluidly couples individual ones of the plurality of screen elements to individual ones of the fluid inlets.

53. The screen assembly of example 52 wherein the fluid space fluidly couples the plurality of screen elements to the fluid inlets.

54. A system for shunting fluid, the system comprising:
a housing;
a plate assembly within the housing, the plate assembly including—
   a plurality of fluid inlets;
   a chamber fluidly coupled to at least one of the fluid inlets, and
   an actuator positioned within the chamber and configured to control the flow of fluid through the system; and
a screen at least partially aligned with a first portion of the system and configured to at least partially prevent debris from entering at least a second portion of the system.

55. The system of example 54 wherein the first portion of the system includes the plate assembly, the chamber, the actuator, or the plurality of fluid inlets.

56. The system of example 54 or example 55 wherein the second portion of the system includes the plurality of fluid inlets, the plate assembly, or the chamber.

57. The system of any of examples 54-56 wherein the screen includes a plurality of screening elements, wherein one or more of the plurality of screening elements are at least partially aligned with the first portion of the system.

58. The system of example 57 wherein each of the plurality of screening elements includes a pore formed in the filter, the individual pores being configured to at least partially prevent debris from entering at least the second portion of the shunting system.

59. The system of example 57 or example 58 wherein each of the screening elements has a width between 0.1 μm and 100 μm

60. The system of any of examples 57-59 wherein each of the plurality of screening elements has a width of about 10 μm.

61. The system of any of examples 57-60 wherein each of the plurality of screening elements has a circular, oval, square, pentagonal, hexagonal, curvilinear, or rectilinear shape.

62. The system of any of examples 54-61 wherein the screen further includes an actuator access region aligned at least partially with the actuator.

63. The system of example 62 wherein the actuator access region (i) is an aperture formed in the filter or (ii) is a portion of the screen that is at least partially transparent.

64. The system of example 62 or example 63 wherein the actuator access region is configured to allow the actuator to be accessible to non-invasive ablative energy.

65. The system of any of examples 54-64 wherein the system is configured (i) to receive first non-invasive ablative energy to at least partially remove debris from the screen and (ii) to receive second non-invasive ablative energy to transition the actuator between a first position and a second position.

66. The system of any of examples 54-65 wherein the actuator is a shape-memory actuator.

67. The system of any of examples 54-66 wherein the screen is formed at least partially from silicon, acrylic, or a shape-memory material.

68. The system of any of examples 54-67 wherein the screen is formed at least partially from polydimethylsiloxane (PDMS), polydimethylacrylamide (PDMA), or super-elastic nitinol.

69. The system of any of examples 54-68 wherein the plurality of fluid inlets includes a first fluid inlet, the chamber is a first chamber fluidly coupled to the first fluid inlet, and the actuator is a first actuator, and wherein:
the plurality of fluid inlets includes a second fluid inlet;
the plate assembly includes—
   a second chamber fluidly coupled to the second fluid inlet, and
   a second actuator positioned within the second chamber and configured to control the flow of fluid through the system; and
the screen is at least partially aligned with a third portion of the system and configured to at least partially prevent debris from entering at least a fourth portion of the system,

70. The system of example 69 wherein—
the third portion includes the plate assembly, the second chamber, the second actuator, or the second fluid inlet, and
the fourth portion includes the second fluid inlet, the plate assembly, or the second chamber.

71. The system of any of examples 54-70 wherein the screen includes a sealing element configured to sealingly engage with the plate assembly to form a substantially fluid-impermeable seal therewith.

72. The system of example 71 wherein the sealing element is configured to extend at least partially around individual ones of the plurality of fluid inlets.

73. The system of example 71 or example 72 wherein the sealing element is a first sealing element, wherein the screen includes a second sealing element, and wherein the plurality of fluid inlets includes a first fluid inlet and a second fluid inlet, and wherein:
the first sealing element sealingly engages the plate assembly around the first inlet; and
the second sealing element sealingly engages the plate assembly around the second inlet.

74. The system of any of examples 71-73 wherein the sealing element extends outwardly from the screen toward the plate assembly.

75. The system of any of examples 71-74 wherein the plate assembly includes a recess configured to receive the sealing element.

76. The system of any of examples 54-75 wherein the screen defines a fluid space between the screen and the plate assembly.

77. The system of example 76 wherein the screen includes a plurality of screening elements, and wherein the fluid space fluidly couples individual ones of the plurality of screen elements to individual ones of the fluid inlets.

78. The system of example 77 wherein the fluid space fluidly couples the plurality of screen elements to the fluid inlets.

79. The system of any of examples 54-78, further comprising a channel fluidly coupled to one or more of the fluid inlets and configured to receive fluid therefrom.

80. The system of example 79 wherein the channel has a first end and a second end opposite the first end, and wherein the first end of the channel has a first dimension, and further wherein the second end of the channel has a second dimension different than the first end.

81. The system of example 80 wherein the first dimension is a first width and the second dimension is a second width greater than the first width.

82. The system of example 80 or example 81 wherein the first dimension is a first cross-sectional area and the second dimension is a second cross-sectional area greater than the first cross-sectional area.

83. The system of any of examples 79-82 wherein each of the plurality of fluid inlets are fluidly coupled to the channel in series.

84. The system of example 83 wherein the plurality of fluid inlets includes a first fluid inlet and a second fluid inlet, and wherein the second fluid inlet is positioned downstream from the first fluid inlet, and further wherein the actuator is configured to control the flow of fluid the second fluid inlet.

85. A method for operating a shunting system, the method comprising:
directing non-invasive ablative energy toward a screen assembly of the shunting system;
removing at least a portion of debris from a screen of the screen assembly; and
transitioning an actuator of the adjustable shunting system between a first position and a second position.

86. The method of example 85 wherein directing the non-invasive ablative energy toward the screen assembly includes applying the non-invasive ablative energy to at least a portion of the screen.

87. The method of example 85 or example 86 wherein directing the non-invasive ablative energy toward the screen assembly includes applying the non-invasive ablative energy to a first end portion or a second end portion of the screen.

88. The method of any of examples 85-87 wherein directing the non-invasive ablative energy toward the screen assembly includes applying the non-invasive ablative energy to one or more screening elements of the screen.

89. The method of any of examples 85-88 wherein directing the non-invasive ablative energy toward the screen assembly includes directing the non-invasive ablative energy toward one or more actuator access regions of the screen.

90. The method of example 89 wherein directing the non-invasive ablative energy toward the one or more actuator access regions includes applying the non-invasive ablative energy to one or more actuators of the shunting system via the one or more actuator access regions, wherein each actuator is at least partially aligned with one of the one or more actuator access regions.

91. The method of example 89 or example 90 wherein directing the non-invasive ablative energy toward the one or more actuator access regions includes applying the non-invasive ablative energy to one or more actuation element target regions of one or more actuators of the shunting system via the one or more actuator access regions, wherein each actuation element target region is at least partially aligned with one of the one or more actuator access regions.

92. The method of any of examples 85-91 wherein directing the non-invasive ablative energy toward the screen assembly includes:
applying first non-invasive ablative energy to the screen; and
directing second non-invasive ablative energy toward one or more actuator access regions of the screen assembly.

93. The method of example 92 wherein the first non-invasive ablative energy includes a first laser energy, the second non-invasive ablative includes a second laser energy, and wherein the first non-invasive ablative energy and the second non-invasive ablative energy have a same optical property.

94. The method of example 92 or example 93 wherein the first non-invasive ablative energy includes a first laser energy, the second non-invasive ablative includes a second laser energy, and wherein the second non-invasive ablative energy has a different optical property than the first non-invasive ablative energy.

95. The method of any of examples 85-94 wherein removing at least the portion of the debris from the screen includes removing at least the portion of the debris from one or more screening elements of the screen.

96. The method of any of examples 85-95 wherein removing at least the portion of the debris from the screen includes at least partially dissolving or burning-off the debris from the screen.

### CONCLUSION

The above detailed description of embodiments of the technology are not intended to be exhaustive or to limit the technology to the precise form disclosed above. Although specific embodiments of, and examples for, the technology are described above for illustrative purposes, various equivalent modifications are possible within the scope of the technology as those skilled in the relevant art will recognize. For example, any of the features of the adjustable shunting systems and/or screen assemblies described herein may be combined with any of the features of the other adjustable shunting systems and/or screen assemblies herein and vice versa. Moreover, although steps are presented in a given order, alternative embodiments may perform steps in a different order. The various embodiments described herein may also be combined to provide further embodiments.

From the foregoing, it will be appreciated that specific embodiments of the technology have been described herein for purposes of illustration, but well-known structures and functions associated with adjustable shunting systems have not been shown or described in detail to avoid unnecessarily obscuring the description of the embodiments of the technology. Where the context permits, singular or plural terms may also include the plural or singular term, respectively.

Unless the context clearly requires otherwise, throughout the description and the examples, the words “comprise,” “comprising,” and the like are to be construed in an inclusive sense, as opposed to an exclusive or exhaustive sense: that is to say, in the sense of “including, but not limited to.” As used herein, the terms “connected,” “coupled,” or any variant thereof, means any connection or coupling, either direct or indirect, between two or more elements; the coupling of connection between the elements can be physical, logical, or a combination thereof. Additionally, the words “herein,” “above,” “below,” and words of similar import, when used in this application, shall refer to this application as a whole and not to any particular portions of this application. Where the context permits, words in the above Detailed Description using the singular or plural number may also include the plural or singular number respectively. As used herein, the phrase “and/or” as in “A and/or B” refers to A alone, B alone, and A and B. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with some embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

I/We claim:

1. A system for shunting fluid from a first body region to a second body region within a patient, the system comprising:
a first layer including a first fluid inlet and a second fluid inlet, wherein the first and second fluid inlets are each configured to receive fluid from the first body region;
a second layer coupled to the first layer and including a first fluid outlet and a second fluid outlet, wherein the first and second fluid outlets are each configured to be positioned within the second body region, and wherein the first fluid outlet is fluidly coupled to the first fluid inlet via a first channel and the second fluid outlet is fluidly coupled to the second fluid inlet via a second channel; and
an actuator positioned to selectively control the flow of fluid from the first fluid inlet of the first layer into the first channel of the second layer,
wherein, independent of a state of the actuator, the second channel is configured to receive fluid from the second fluid inlet.

2. The system of claim 1 wherein the first fluid outlet and the second fluid outlet are positioned at a distal end of the second layer, and wherein the first fluid outlet is spaced apart and discrete from the second fluid outlet.

3. The system of claim 1 wherein the first layer defines a chamber configured to receive fluid from the first body region, and wherein the actuator is positioned within the chamber and configured to adjust the flow of fluid within the chamber through the first fluid inlet.

4. The system of claim 1 wherein the first layer includes (i) a third fluid outlet fluidly coupled to one or both of the first channel and the second channel and (ii) a fluid reservoir positioned downstream from the one or both of the first fluid inlet and the second fluid inlet and upstream from the third fluid outlet, and wherein the fluid reservoir is configured to substantially prevent fluid flow through the third fluid outlet until the fluid reservoir is at least partially filled with fluid received from one or both of the first channel and the second channel.

5. The system of claim 4 wherein the third fluid outlet is fluidly coupled to the fluid reservoir by a third channel extending between the fluid reservoir and the third fluid outlet.

6. The system of claim 1, further comprising a third layer including a third fluid outlet fluidly coupled to one or both of the first channel and the second channel.

7. The system of claim 6 wherein the first layer includes a first side and a second side opposite the first side, and wherein (i) the second layer is coupled to the first side, and (ii) the third layer is coupled to the second side.

8. The system of claim 6 wherein the first layer, the second layer, and the third layer are configured so that fluid flows (i) from the first layer to the second layer in a first direction perpendicular to a longitudinal axis of the system and (ii) from the second layer through the first layer to the third layer in a second direction opposite the first direction and perpendicular to the longitudinal axis.

9. The system of claim 6 wherein first layer includes a fluid reservoir configured to fluidly couple one or both of the first channel and the second channel to the third fluid outlet.

10. The system of claim 9 wherein the first layer includes a third channel configured to fluidly couple the fluid reservoir and the third fluid outlet.

11. The system of claim 10 wherein at least a first portion of one or both of the first channel and the second channel are configured to direct fluid flow in a first direction, and wherein at least a second portion of the third channel is configured to direct fluid flow in a second direction opposite the first direction.

12. The system of claim 1 wherein, independent of a state of the actuator, the first channel is configured to receive fluid from the first fluid inlet.

13. The system of claim 1 wherein the actuator is configured to transition between (i) a first position in which the actuator allows fluid to flow between the first fluid inlet and the first fluid outlet at a first rate and (ii) a second position in which the actuator allows fluid to flow between the first fluid inlet and the first fluid outlet at a second rate less than the first rate.

14. A system for shunting fluid from a first body region to a second body region within a patient, the system comprising:
a housing at least partially defining a channel configured to allow fluid flow from the first body region to the second body region, wherein the channel includes—
a first inlet, and
a second inlet positioned downstream from the first inlet; and
an actuator positioned to control the flow of fluid through the second inlet, wherein the actuator is configured to transition between a first position and a second position,
wherein—
when the actuator is in the first position, the channel provides a first resistance to fluid flow therethrough, and
when the actuator is in the second position, the channel provides a second resistance to fluid flow therethrough, the second resistance greater than the first resistance.

15. The system of claim 14 wherein:
when the actuator is in the first position, at least a portion of the actuator is offset from second inlet; and
when the actuator is in the second position, the portion of the actuator is at least partially aligned with the second inlet.

16. The system of claim 14 wherein:
when the actuator is in the first position, the actuator allows fluid to flow through the second inlet at a first rate; and
when the actuator is in the second position, the actuator allows fluid to flow through the second inlet at a second rate less than the first rate.

17. The system of claim 14 wherein, independent of a position of the actuator, the first inlet is configured to allow fluid to flow into the channel.

18. The system of claim 14 wherein, when the actuator is in the first position, substantially all fluid flow into the channel is via the second inlet.

19. The system of claim 14 wherein the housing comprises a first layer and a second layer coupled to the first layer.

20. The system of claim 19 wherein the first layer at least partially defines the channel, and wherein the second layer at least partially defines a chamber configured to receive the actuator.

21. A system for shunting fluid from a first body region to a second body region within a patient, the system comprising:
a housing including a plurality of fluid inlets, wherein individual ones of the plurality of fluid inlets include a widened middle portion;
a plate assembly within the housing, the plate assembly including—
a chamber fluidly coupled to at least one of the fluid inlets, and
an actuator positioned within the chamber and configured to selectively control the flow of fluid through the system.

22. The system of claim 21 wherein the plurality of fluid inlets is a plurality of first fluid inlets, the system further comprising a plurality of second inlets, wherein individual ones of the plurality of second inlets include an angled middle portion.

23. The system of claim 22, further comprising a channel fluidly coupled to the plate assembly, wherein the actuator is positioned to selectively control the flow of fluid through the channel, and wherein individual ones of the plurality of second inlets are fluidly coupled to the channel.

24. The system of claim 23, further comprising a fluid reservoir, wherein the fluid reservoir fluidly couples individual ones of the plurality of second inlets to the channel.

25. The system of claim 23 wherein the channel has a first end and a second end opposite the first end, and wherein the first end of the channel has a first dimension, and the second end of the channel has a second dimension different than the first end.

26. The system of claim 25 wherein the first dimension is a first width, wherein the second dimension is a second width and the second width is greater than the first width.

27. The system of claim 25 wherein the first dimension is a first cross-sectional area and the second dimension is a second cross-sectional area, and wherein the second cross-sectional area is greater than the first cross-sectional area.

28. A system for shunting fluid from a first body region to a second body region within a patient, the system comprising:
a housing;
a plate assembly within the housing, the plate assembly including—
a chamber,
a plurality of fluid inlets positioned within the chamber,
a flow channel, wherein each of the plurality of fluid inlets are fluidly coupled to the flow channel, and
an actuator positioned within the chamber and configured to control the flow of fluid through the flow channel.

29. The system of claim 28 wherein each of the plurality of fluid inlets are fluidly coupled to the channel in series.

30. The system of claim 28 wherein the plurality of fluid inlets include a first fluid inlet and a second fluid inlet, and wherein the second fluid inlet is positioned downstream from the first fluid inlet, and further wherein the actuator is configured to control the flow of fluid the second fluid inlet.

31. The system of claim 28 wherein the flow channel has a first end and a second end opposite the first end, and wherein the first end of the flow channel has a first dimension and the second end of the flow channel has a second dimension different than the first end.

32. The system of claim 31 wherein the first dimension is a first width and the second dimension is a second width, and wherein the second width is greater than the first width.

33. The system of claim 31 wherein the first dimension is a first cross-sectional area and the second dimension is a second cross-sectional area, and wherein the second cross-sectional area is greater than the first cross-sectional area.

34. A screen assembly for use with an adjustable shunting system for treating a patient, the screen assembly comprising:
a screen having a first end portion and a second end portion spaced apart from the first end portion,
wherein the first end portion of the screen is at least partially aligned with fluid inlets of the shunting system, and
wherein, during operation, the screen is configured to (i) at least partially prevent debris from entering the fluid inlets of the shunting system, and (ii) receive non-invasive ablative energy at the first end portion to at least partially remove the debris from the screen.

35. The screen assembly of claim 34 wherein the screen further includes one or more actuator access regions positioned at least partially between the first end portion of the screen and the second end portion of the screen.

36. The screen assembly of claim 35 wherein each of the actuator access regions is at least partially aligned with at least one actuator of the shunting system.

37. The screen assembly of claim 35 wherein each of the one or more actuator access regions is (i) an aperture formed in the screen or (ii) at least partially transparent.

38. The screen assembly of claim 34 wherein:
the first end portion includes a plurality of screening elements, and
each of the fluid inlets is at least partially aligned with one or more of the plurality of screening elements.

39. The screen assembly of claim 38 wherein each of the plurality of screening elements includes a pore formed in the screen, the individual pores being configured to at least partially prevent debris from entering the fluid inlets of the shunting system.

40. The screen assembly of claim 38 wherein each of the plurality of screening elements has a width between 0.1 μm and 100 μm.

41. The screen assembly of claim 38 wherein each of the plurality of screening elements has a width of 10 μm.

42. The screen assembly of claim 38 wherein each of the plurality of screening elements has a circular, oval, square, pentagonal, hexagonal, curvilinear, or rectilinear shape.

43. The screen assembly of claim 38 wherein the plurality of screening elements is a plurality of first screening elements and the plurality of fluid inlets is a plurality of first fluid inlets, wherein:
the second end portion of the screen is at least partially aligned with one or more second fluid inlets of the shunting system;
the second end portion further includes a plurality of second screening elements; and
each of the second fluid inlets is at least partially aligned with one or more of the plurality of second screening elements.

44. The screen assembly of claim 43 wherein each of the plurality of second screening elements includes a pore formed in the second end portion of the screen.

45. The screen assembly of claim 43 wherein the first screening elements and the second screening elements have the same dimension.

46. The screen assembly of claim 43 wherein the first screening elements and the second screening elements have the same shape.

47. The screen assembly of claim 34 wherein the screen is formed at least partially from polydimethylsiloxane (PDMS), polydimethylacrylamide (PDMA), or super-elastic nitinol.

48. The screen assembly of claim 34, further comprising a sealing element configured to sealingly engage with the adjustable shunting system to form a substantially fluid-impermeable seal therewith.

49. The screen assembly of claim 48 wherein the sealing element is configured to extend at least partially around the fluid inlets.

50. The screen assembly of claim 48 wherein the sealing element extends outwardly away from the screen.

51. The screen assembly of claim 34 wherein the screen defines a fluid space between the screen and the fluid inlets.

52. The screen assembly of claim 51 wherein the screen includes a plurality of screening elements, and wherein the fluid space fluidly couples individual ones of the plurality of screen elements to individual ones of the fluid inlets.

53. The screen assembly of claim 52 wherein the fluid space fluidly couples the plurality of screen elements to the fluid inlets.

54. A system for shunting fluid, the system comprising:
a housing;
a plate assembly within the housing, the plate assembly including—
a plurality of fluid inlets;
a chamber fluidly coupled to at least one of the fluid inlets, and
an actuator positioned within the chamber and configured to control the flow of fluid through the system; and
a screen at least partially aligned with a first portion of the system and configured to at least partially prevent debris from entering at least a second portion of the system.

55. The system of claim 54 wherein the first portion of the system includes the plate assembly, the chamber, the actuator, or the plurality of fluid inlets.

56. The system of claim 54 wherein the second portion of the system includes the plurality of fluid inlets, the plate assembly, or the chamber.

57. The system of claim 54 wherein the screen includes a plurality of screening elements, wherein one or more of the plurality of screening elements are at least partially aligned with the first portion of the system.

58. The system of claim 57 wherein each of the plurality of screening elements includes a pore formed in the filter, the individual pores being configured to at least partially prevent debris from entering at least the second portion of the shunting system.

59. The system of claim 57 wherein each of the screening elements has a width between 0.1 μm and 100 μm.

60. The system of claim 57 wherein each of the plurality of screening elements has a width of about 10 μm.

61. The system of claim 57 wherein each of the plurality of screening elements has a circular, oval, square, pentagonal, hexagonal, curvilinear, or rectilinear shape.

62. The system of claim 54 wherein the screen further includes an actuator access region aligned at least partially with the actuator.

63. The system of claim 62 wherein the actuator access region (i) is an aperture formed in the filter or (ii) is a portion of the screen that is at least partially transparent.

64. The system of claim 62 wherein the actuator access region is configured to allow the actuator to be accessible to non-invasive ablative energy.

65. The system of claim 54 wherein the system is configured (i) to receive first non-invasive ablative energy to at least partially remove debris from the screen and (ii) to receive second non-invasive ablative energy to transition the actuator between a first position and a second position.

66. The system of claim 54 wherein the actuator is a shape-memory actuator.

67. The system of claim 54 wherein the screen is formed at least partially from silicon, acrylic, or a shape-memory material.

68. The system of claim 54 wherein the screen is formed at least partially from polydimethylsiloxane (PDMS), polydimethylacrylamide (PDMA), or super-elastic nitinol.

69. The system of claim 54 wherein the plurality of fluid inlets includes a first fluid inlet, the chamber is a first chamber fluidly coupled to the first fluid inlet, and the actuator is a first actuator, and wherein:
the plurality of fluid inlets includes a second fluid inlet;
the plate assembly includes—
a second chamber fluidly coupled to the second fluid inlet, and
a second actuator positioned within the second chamber and configured to control the flow of fluid through the system; and
the screen is at least partially aligned with a third portion of the system and configured to at least partially prevent debris from entering at least a fourth portion of the system.

70. The system of claim 69 wherein—
the third portion includes the plate assembly, the second chamber, the second actuator, or the second fluid inlet, and
the fourth portion includes the second fluid inlet, the plate assembly, or the second chamber.

71. The system of claim 54 wherein the screen includes a sealing element configured to sealingly engage with the plate assembly to form a substantially fluid-impermeable seal therewith.

72. The system of claim 71 wherein the sealing element is configured to extend at least partially around individual ones of the plurality of fluid inlets.

73. The system of claim 71 wherein the sealing element is a first sealing element, wherein the screen includes a second sealing element, and wherein the plurality of fluid inlets includes a first fluid inlet and a second fluid inlet, and wherein:
the first sealing element sealingly engages the plate assembly around the first inlet; and
the second sealing element sealingly engages the plate assembly around the second inlet.

74. The system of claim 71 wherein the sealing element extends outwardly from the screen toward the plate assembly.

75. The system of claim 71 wherein the plate assembly includes a recess configured to receive the sealing element.

76. The system of claim 54 wherein the screen defines a fluid space between the screen and the plate assembly.

77. The system of claim 76 wherein the screen includes a plurality of screening elements, and wherein the fluid space fluidly couples individual ones of the plurality of screen elements to individual ones of the fluid inlets.

78. The system of claim 77 wherein the fluid space fluidly couples the plurality of screen elements to the fluid inlets.

79. The system of claim 54, further comprising a channel fluidly coupled to one or more of the fluid inlets and configured to receive fluid therefrom.

80. The system of claim 79 wherein the channel has a first end and a second end opposite the first end, and wherein the first end of the channel has a first dimension, and further wherein the second end of the channel has a second dimension different than the first end.

81. The system of claim 80 wherein the first dimension is a first width and the second dimension is a second width greater than the first width.

82. The system of claim 80 wherein the first dimension is a first cross-sectional area and the second dimension is a second cross-sectional area greater than the first cross-sectional area.

83. The system of claim 79 wherein each of the plurality of fluid inlets are fluidly coupled to the channel in series.

84. The system of claim 83 wherein the plurality of fluid inlets includes a first fluid inlet and a second fluid inlet, and wherein the second fluid inlet is positioned downstream from the first fluid inlet, and further wherein the actuator is configured to control the flow of fluid the second fluid inlet.

85. A method for operating a shunting system, the method comprising:
directing non-invasive ablative energy toward a screen assembly of the shunting system;
removing at least a portion of debris from a screen of the screen assembly; and
transitioning an actuator of the adjustable shunting system between a first position and a second position.

86. The method of claim 85 wherein directing the non-invasive ablative energy toward the screen assembly includes applying the non-invasive ablative energy to at least a portion of the screen.

87. The method of claim 85 wherein directing the non-invasive ablative energy toward the screen assembly includes applying the non-invasive ablative energy to a first end portion or a second end portion of the screen.

88. The method of claim 85 wherein directing the non-invasive ablative energy toward the screen assembly includes applying the non-invasive ablative energy to one or more screening elements of the screen.

89. The method of claim 85 wherein directing the non-invasive ablative energy toward the screen assembly includes directing the non-invasive ablative energy toward one or more actuator access regions of the screen.

90. The method of claim 89 wherein directing the non-invasive ablative energy toward the one or more actuator access regions includes applying the non-invasive ablative energy to one or more actuators of the shunting system via the one or more actuator access regions, wherein each actuator is at least partially aligned with one of the one or more actuator access regions.

91. The method of claim 89 wherein directing the non-invasive ablative energy toward the one or more actuator access regions includes applying the non-invasive ablative energy to one or more actuation element target regions of one or more actuators of the shunting system via the one or more actuator access regions, wherein each actuation element target region is at least partially aligned with one of the one or more actuator access regions.

92. The method of claim 85 wherein directing the non-invasive ablative energy toward the screen assembly includes:
applying first non-invasive ablative energy to the screen; and
directing second non-invasive ablative energy toward one or more actuator access regions of the screen assembly.

93. The method of claim 92 wherein the first non-invasive ablative energy includes a first laser energy, the second non-invasive ablative includes a second laser energy, and wherein the first non-invasive ablative energy and the second non-invasive ablative energy have a same optical property.

94. The method of claim 92 wherein the first non-invasive ablative energy includes a first laser energy, the second non-invasive ablative includes a second laser energy, and wherein the second non-invasive ablative energy has a different optical property than the first non-invasive ablative energy.

95. The method of claim 85 wherein removing at least the portion of the debris from the screen includes removing at least the portion of the debris from one or more screening elements of the screen.

96. The method of claim 85 wherein removing at least the portion of the debris from the screen includes at least partially dissolving or burning-off the debris from the screen.
